(19) United States
(12) Patent Application Publication
(10) Pub. No.: US 2026/0218135 A1
(43) Pub. Date: Jul. 30, 2026

(54) USE OF RTL8-DEFICIENT CELLS TO PRODUCE BIOCOMPATIBLE, PEG10-DERIVED VIRUS-LIKE PARTICLES (VLP)

(71) Applicant: The Regents of the University of Colorado, a body corporate, Denver, CO (US)

(72) Inventors: Alexandra Whiteley, Boulder, CO (US); William Campodonico-Burnett, Boulder, CO (US); Aaron Whiteley, Boulder, CO (US)

(21) Appl. No.: 19/142,176

(22) PCT Filed: Dec. 15, 2023
(86) PCT No.: PCT/US2023/084278

§ 371 Date: Jun. 20, 2025

Related U.S. Application Data
(60) Provisional application No. 63/434,125, filed on Dec. 21, 2022.
(60) Provisional application No. 63/536,006, filed on Aug. 31, 2023.

(51) Int. Cl. C12N 7/00; C07K 14/47; C12N 5/071

(52) U.S. Cl. CPC C12N 7/00; C07K 14/4702; C12N 5/0602; C12N 2510/00; C12N 2770/00023

(57) ABSTRACT

The present invention is directed to novel systems, methods, and compositions for increasing the production of virus-like particles (VLPs) in a cell. In particular, the invention includes disrupting the activity or expression of RTL8 and/or UBQLN2 in the cell, which causes an increase in PEG10 VLP formation.

### CROSS-REFERENCE TO RELATED APPLICATIONS

This International PCT application claims the benefit of and priority to U.S. Provisional Application No. 63/434,125, filed Dec. 21, 2022, and U.S. Provisional Application No. 63/536,006, filed Aug. 31, 2023, the specification, claims and drawings of which are incorporated herein by reference in their entirety.

### SEQUENCE LISTING

The instant application contains contents of the electronic sequence listing (90245.00922-Sequence-Listing.xml; Size: 16,842 bytes; and Date of Creation: Dec. 13, 2023) is herein incorporated by reference in its entirety.

### TECHNICAL FIELD

The present invention is directed to novel systems, methods, and compositions for increasing the production of virus-like particles (VLPs) in a cell, and in particular PEG10 VLPs.

### BACKGROUND

Transposable elements (TEs), which include DNA transposons and RNA retrotransposons, are virus-like genetic elements capable of replicating within the genome through a variety of complex mechanisms. Due to the catastrophic consequences of unchecked genomic integration, eukaryotes have developed extensive networks to restrict transposable element expression and activity at multiple stages of their lifecycles. These mechanisms of restriction act at transcriptional, translational, and post-translational stages of the transposon lifecycle, and are found in diverse eukaryotes from yeast to humans. Inhibition or loss of these restriction factors results in elevated transposable element activity, which can result in genomic instability and human disease. Rather than being restricted by host factors, expression of a subset of TE-derived genes is essential for organismal health. These genes are referred to as ‘domesticated’ and have a variety of adaptive roles, including the maintenance of genomic integrity, development, and even viral restriction. However, despite the selective pressure towards host fitness, recent studies have shown that a subset of these domesticated elements retain pathological effects and can directly contribute to human disease. Therefore, it would be advantageous for the host to use the same, or repurposed TE restriction factors to regulate their domesticated counterparts. However, to date no such restriction factors of domesticated elements have been identified.

The domesticated retrotransposon Paternally Expressed Gene 10 (PEG10) embodies a balance between adaptation and pathology: PEG10 is necessary for placentation in mice but also drives cancer and neurological disease in humans. The mechanism of PEG10-mediated adaptive and pathological roles remains poorly understood but may involve the protein's rare ability to form virus-like particles (VLPs) that are exported from the cell. For other domesticated genes such as Arc, formation and release of a VLP is essential to its adaptive function in the organism; for PEG10, VLP formation may contribute to either adaptive or pathological roles.

Here, the present inventors describe a multifaceted regulatory network that antagonizes PEG10 VLP formation and release from human cells. It was recently discovered that the proteasome shuttle factor ubiquilin 2 (UBQLN2) selectively targets PEG10 for degradation, and here show that this translates to decreased VLP production. The present inventors also describe a novel role for the human gene RTL8, a domesticated retrotransposon with structural similarity to the N-terminal lobe of PEG10 gag, in the restriction of PEG10-derived VLPs. RTL8 binds to the PEG10 N-terminal lobe, is found in PEG10 VLPs, and causes PEG10 to be retained in the cytosol. Together, these findings suggest a model in which RTL8 unproductively incorporates into PEG10 VLPs, thereby inhibiting their assembly or release. RTL8's restriction of PEG10 appears to be species-specific, suggesting that the two domesticated retroelements co-evolved in conflict. These results illuminate a new role for RTL8 in the inhibition of PEG10 activity and may have important implications for reproductive and neurological health. These results also identify a novel conflict between two domesticated retrotransposons in the human genome and highlight the unique demands of balancing beneficial and detrimental roles of TEs.

### SUMMARY OF THE INVENTION

As described by Segel M., at al., a unique feature of PEG10 is its ability to readily form virus-like particles (VLPs) which may contribute to its adaptive and pathological nature, is being investigated as a source of human biocompatible VLPs for the delivery of mRNA-based vaccines, gene therapies or other biological compounds. For example, cells expressing PEG10+a target inhibitory mRNA could be grown in vitro to generate high quantities of VLPs for the generation of vaccines. However, the cellular regulatory systems that govern PEG10-derived VLP assembly remain unknown.

Here, the present inventors describe restriction of PEG10 VLP abundance via UBQLN2 and the poorly characterized domesticated retrotransposon RTL8. The gag-like RTL8 antagonizes PEG10 through incorporation into VLPs in a manner reminiscent of transposable element inhibitors from diverse eukaryotes. These results represent the first known instance of a retroelement-derived restriction factor targeting another domesticated retrotransposon and have implications for the study of PEG10-mediated disease.

As further described below, the present inventors have identified that the human gene RTL8 encodes a protein which inhibits PEG10-derived virus like particle production in cells. As such, in one embodiment of the present invention, knockdown or knockout of RTL8 in human cells can increase yield of PEG10-derived VLPs for the purposes of vaccine production, or deliver of gene therapies, such as CRISPR or other biological therapeutics.

In another aspect, the invention includes inhibiting the expression or activity of UBQLN2 (SEQ ID NO. 9), which can increase the production of PEG10-derived virus-like particle in a cell.

### BRIEF DESCRIPTION OF DRAWINGS

FIG. 1A-E: PEG10-derived VLPs are naturally released by human cells. a) Representative western blot showing PEG10 abundance in lysate from A549, HepG2, HEK293, hTR-8, and U87 cell lines. n=6 representative experiments, b) Quantification of PEG10 gag (left) and gag-pol (right) abundance in lysate. c) Representative western blot showing PEG10 VLP production across cell lines. n=6 representative experiments, d) Quantification of PEG10 gag (left) and gag-pol (right) abundance in VLPs of human cell lines. e) Iodixanol fractionation of conditioned medium from HepG2 cells. Left: schematic of iodixanol gradient preparation and harvest. CM: conditioned medium. Arrow with 1 indicates where needle was inserted to start fraction collection. Star denotes the percentage of iodixanol which demonstrated the beginning of PEG10 detection. Right: western blot of 13 consecutive fractions from iodixanol gradient showing PEG10, ALIX, and Tubulin. Approximate division of iodixanol fractions is denoted at bottom based on color and flowrate of eluate.

FIG. 2A-F: RTL8 incorporates into PEG10 VLPs and decreases the efficiency of release. a) Schematic of PEG10 with RTL8. b) Representative western blot measuring FLAG-RTL8c presence in the VLP fraction. FLAG-RTL8c was co-expressed without or with HA-PEG10. FLAG-RTL8c release in the VLP fraction is dependent upon co-expression of HA-PEG10. c) Representative western blot showing HA-PEG10 lysate (left) and VLP (right) signal without and with FLAG-RTL8c co-expression. d) Quantification of HA-PEG10 VLP signal with FLAG-RTL8c co-expression. Left: For PEG10 gag-pol overexpression, gag and gag-pol signal was summed to calculate total PEG10 VLP signal. Right: For PEG10 gag overexpression, gag signal was quantified. Significance was determined by T-test. e) Design schematic of a flow assay assessing intracellular PEG10 signal. PEG10 is expressed as a fusion protein with the fluorescent protein Dendra2 at its C-terminus, followed by an IRES-CFP for transfection efficiency control. f) Flow cytometry measurement of intracellular PEG10-Dendra2 abundance. PEG10-Dendra2 was co-expressed with either a control vector (pcDNA3.1), FLAG-RTL8c, or HA-capsidNTD, and PEG10-Dendra2 was measured to determine its intracellular abundance. Data were analyzed by ordinary one-way ANOVA.

FIG. 3A-E: RTL8 interacts with the NTD lobe of PEG10 capsid. a) Crosslinking co-immunoprecipitation of RTL8c with PEG10. HA-tagged PEG10 and FLAG-tagged RTL8c were co-expressed, proteins crosslinked, cells lysed, and complexes immunoprecipitated using an HA antibody. No-HA control cells were transfected only with FLAG-RTL8c before HA-immunoprecipitation. b) Crosslinking co-immunoprecipitation of PEG10 with RTL8c. FLAG-tagged RTL8c and HA-tagged PEG10 were co-expressed and prepared as in FIG. 2b. Complexes were immunoprecipitated using a FLAG antibody or an IgG isotype control antibody. c) Diagram of PEG10 constructs including HA-capsidNTD and HA-capsidCTD truncations tested for interaction with FLAG-RTL8c. N-terminal HA tag is shown in black. d) Representative immunoprecipitations of FLAG-RTL8c showing co-immunoprecipitation of HA-gag and HA-capsidNTD. Asterisk indicates an additional high molecular weight product positive for both HA and FLAG. Due to low relative expression of HA-capsidCTD, an additional experimental replicate was performed with an overabundance of HA-capsidCTD lysate in FIG. S5c. e) Alphafold modeling of RTL8 with PEG10 shows contacts between RTL8 and the CANTD of PEG10. PEG10 is shown in pink, and RTL8 in yellow. Alpha helices of each protein are numbered.

FIG. 4A-F: RTL8 binding and restriction of PEG10 VLP release is species-specific. a) Normalized PEG10-Dendra2 abundance upon control vector, or FLAG-RTL8a, FLAG-RTL8b, or FLAG-RTL8c co-expression. Data were analyzed by ordinary one-way ANOVA with column means compared to mock transfected. b) Alignment of murine and human RTL8 demonstrating high level of conservation. Colors denote amino acid properties. c) Co-immunoprecipitation of murine or human FLAG-RTL8 with HA-tagged PEG10. n=2 independent experiments, d) PEG10-Dendra2 was co-expressed with control, FLAG-HsRTL8c, or FLAG-MmRtl8b and intracellular abundance was measured by flow cytometry. Data were analyzed as shown in FIG. 2f. e) Co-immunoprecipitation of murine or human FLAG-RTL8 with murine HA-tagged Peg10. n=1. f) Murine Peg10-Dendra2 was co-expressed with control, FLAG-HsRTL8c, or FLAG-MmRtl8b and intracellular abundance was measured by flow cytometry. Data were analyzed as in FIGS. 2f and 4d. Data shown are from 3 independent experiments.

FIG. 5: RTL8 incorporates into PEG10 VLPs and decreases the efficiency of release. a) (top) Predicted structure of PEG10 capsid shows the clear separation of NTD and CTD lobes. (bottom) The predicted structure of RTL8, which bears striking similarity to capsidNTD of PEG10.

FIG. 6A-E: Modulation of RTL8 in HepG2 cells is sufficient to alter the production of PEG10-derived VLPs. a) HepG2 cells were transfected with siRNA against RTL8, and conditioned medium was harvested 48 hr later for VLP western blot. n=3 independent experiments, b) Quantitation of RTL8 abundance from FIG. 6a. c-d) Quantification of PEG10 gag (c) or gag-pol (d) abundance in cell lysate. For b-e, data were analyzed by Student's t-test. e) Quantitation of VLP abundance from FIG. 6a. PEG10 VLP signal was measured as the sum of gag-pol and gag band intensities normalized as described in methods.

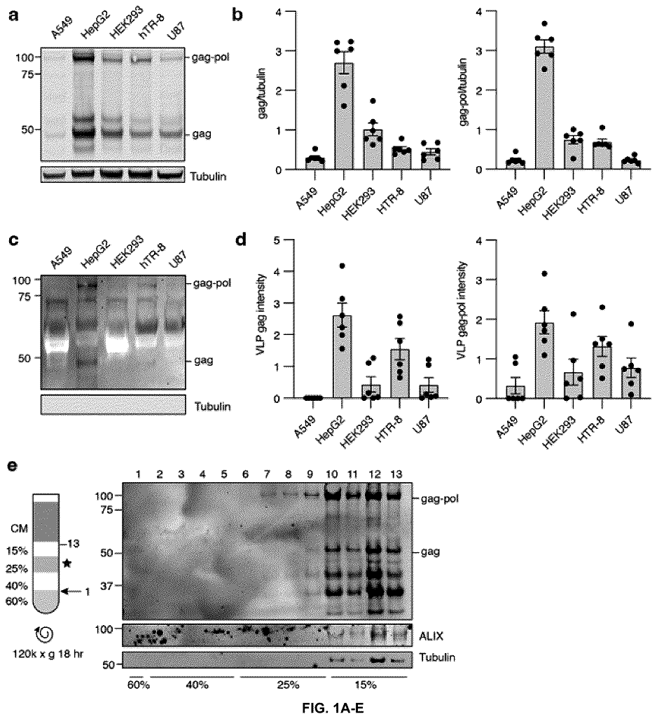

Supplemental FIG. 1A-D: Further quantitation of PEG10 in human cell lines. a-b) Quantification of total PEG10 abundance in lysate (a) and VLP fraction (b) for cells in FIG. 1. Total PEG10 was calculated as the sum of gag-pol and gag signal normalized to tubulin. c) A simple linear regression of PEG10 VLP signal (y axis) and total PEG10 abundance in cell lysate (x axis). d) Quantification of PEG10 frameshift ratio in human cell lines. Frameshift ratio is calculated as gag-pol signal over gag signal. Data are analyzed by ordinary one-way ANOVA. p-values are corrected for multiple comparisons by Dunnett's test.

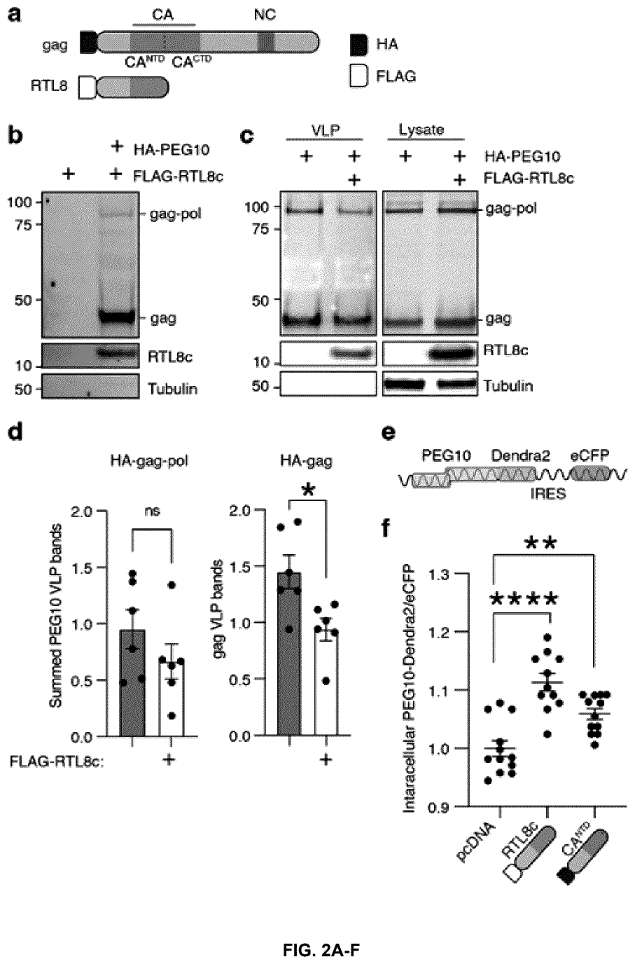

Supplemental FIG. 2: Detection of PEG10-derived VLPs from induced neurons and transfected cells. b) Iodixanol fractionation of media supernatant from HEK293 cells overexpressing HA-PEG10. Left: schematic of iodixanol gradient preparation and harvest. Right: western blot of 13 consecutive fractions from iodixanol gradient showing PEG10, ALIX, and Tubulin.

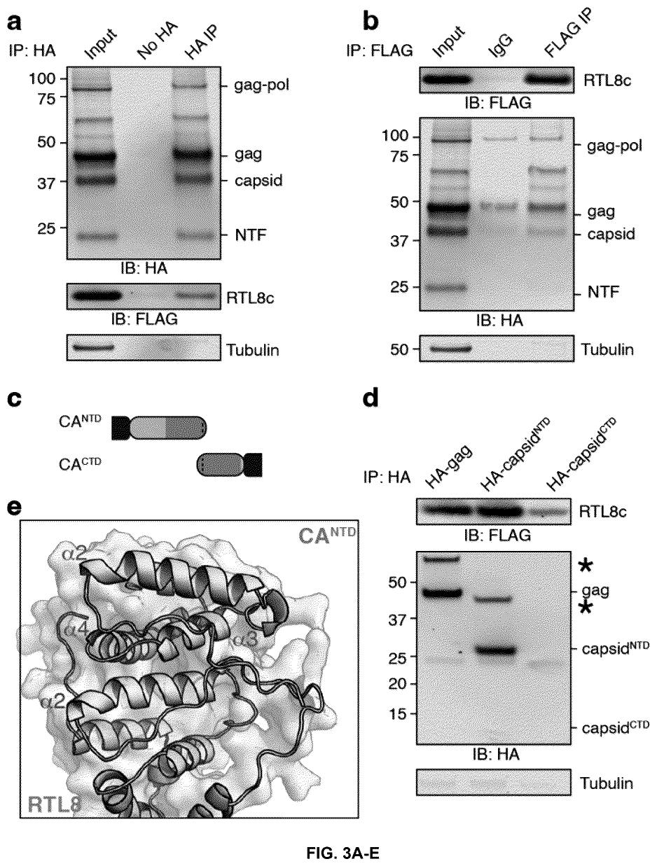

Supplemental FIG. 3A-E: PEG10 VLPs released into media contain a low level of RTL8 incorporation. a) Amino acid alignment of Homo sapiens PEG10 gag with RTL8C. b) PEG10 does not package eGFP into VLPs. HEK293 cells were transfected with eGFP with and without HA-PEG10 and VLPs were isolated. eGFP levels in VLP prep are unaffected by PEG10 expression. c) Iodixanol fractionation of media supernatant from HEK293 cells overexpressing HA-eGFP. Left: schematic of iodixanol gradient preparation and harvest. Right: western blot of 13 consecutive fractions from iodixanol gradient showing eGFP, ALIX, and Tubulin. d) Representative western blots of lysate (left) and VLP (right) samples prepared from cells expressing HA-tagged PEG10 and HA-tagged RTL8c. HA antibody was used to detect proteins, which were identified as either PEG10 or RTL8 based on molecular weight. Shown are data from 4 independent experiments, e) Quantification of HA-RTL8c: HA-PEG10 ratio in lysate and VLP upon co-expression. RTL8 is less abundant in VLPs compared to lysate and is present in VLPs at less than 10% of PEG10 levels on average. Data were analyzed by Student's t-test. ** p<0.01.

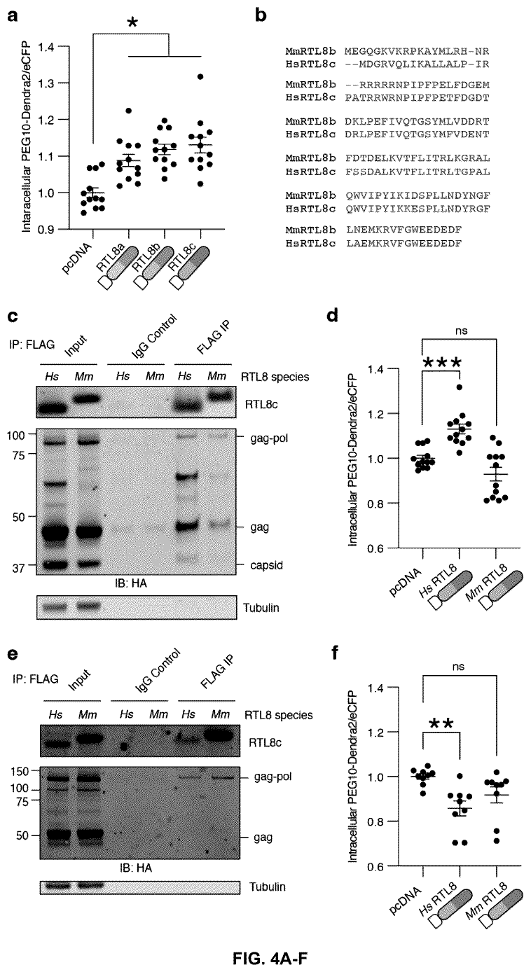

Supplemental FIG. 4A-B: Intracellular PEG10 levels accumulate upon RTL8c co-expression. a) Further quantification of PEG10 abundance in cell lysate with and without RTL8c co-expression. Left: cells transfected with PEG10 gag-pol; both gag and gag-pol bands were used to quantify intracellular PEG10 levels. Right: cells transfected with PEG10 gag. n=5. b) Example western blot demonstrating that PEG10-Dendra2 is capable of forming VLPs that can be recovered from conditioned medium. Cells were transfected with either HA-PEG10 or a construct of PEG10-Dendra2 that only generates gag-pol protein (forced frameshift, ‘FFS’), and VLPs were isolated as previously. Hsp70 band in HA-PEG10 VLP prep reflects contamination.

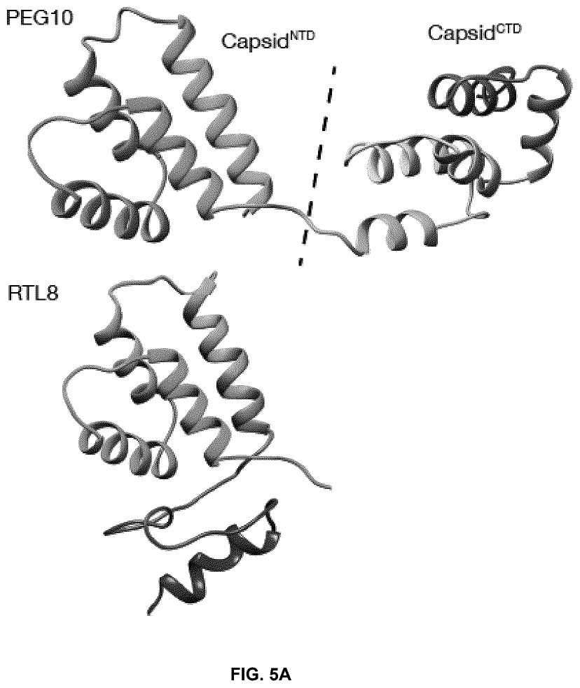

Supplemental FIG. 5A-C: PEG10 closely resembles Ty3 and self-associates in a manner resembling the ancestral retrotransposon. a) Sequence alignment of the Ty3 and PEG10 capsid domains, and full length RTL8c. The demarcation between Ty3 capsidNTD and capsidCTD is indicated with shaded color background. Amino acids are colored by property. b) (top) Structure of the Ty3 capsid. (bottom) Predicted structure of PEG10 gag. c) Co-IP of FLAG-RTL8c with HA-PEG10 gag, capsidNTD, and capsidCTD, as in FIG. 3e. Five times the total input protein was loaded in the capsidCTD immunoprecipitation to detect weak interactions with FLAG-RTL8c that may have been below the detection threshold in FIG. 3e. Input concentrations were high enough that nonspecific interactions with tubulin were detected, but capsidCTD remained below the limit of detection.

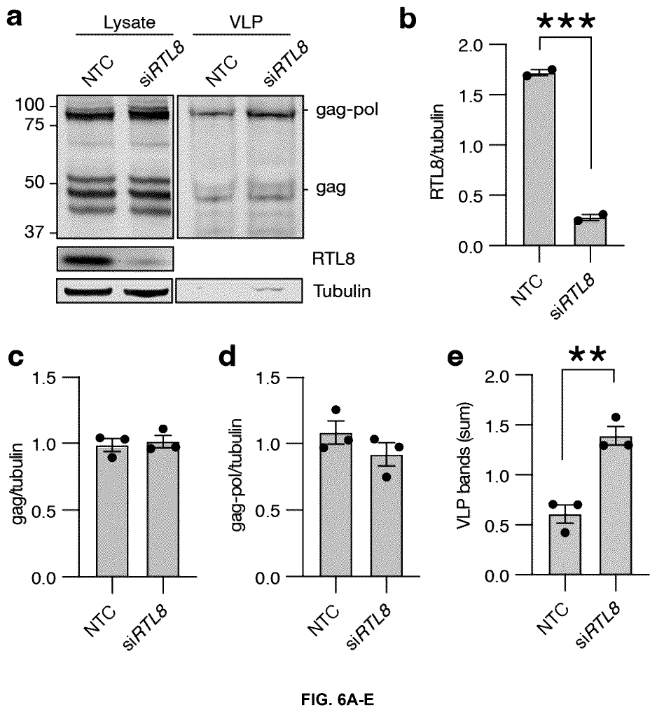

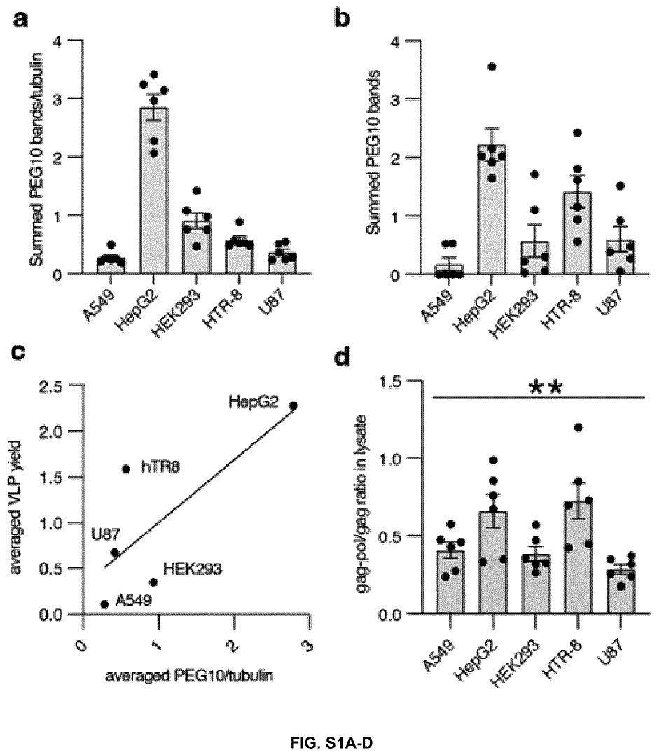

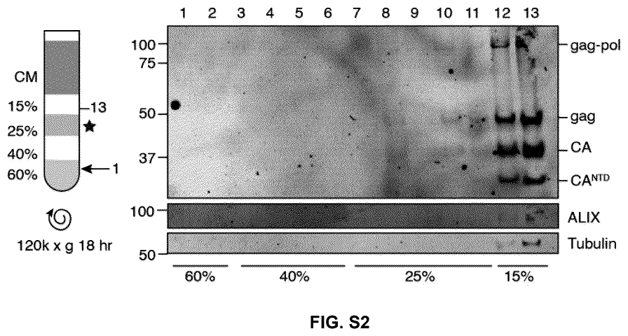

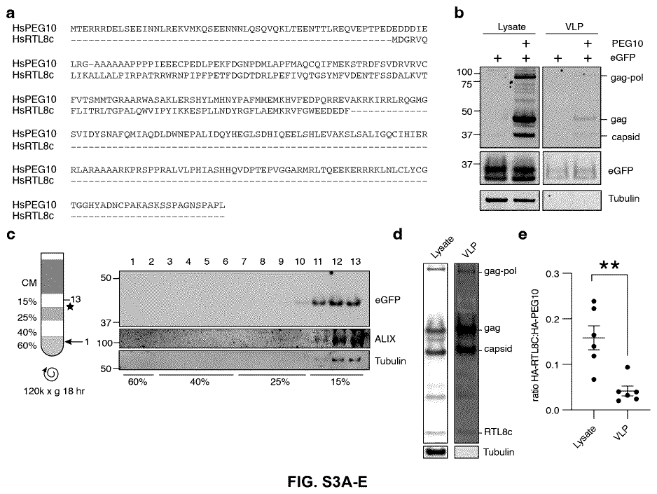

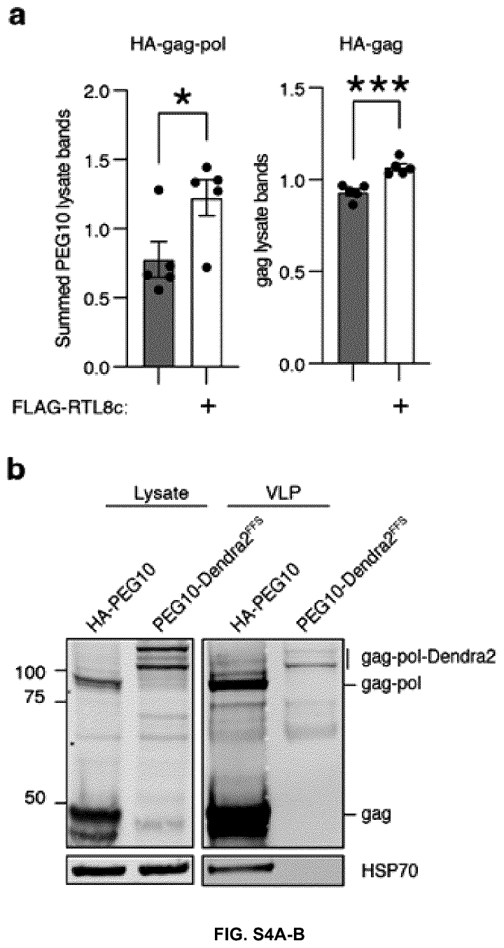

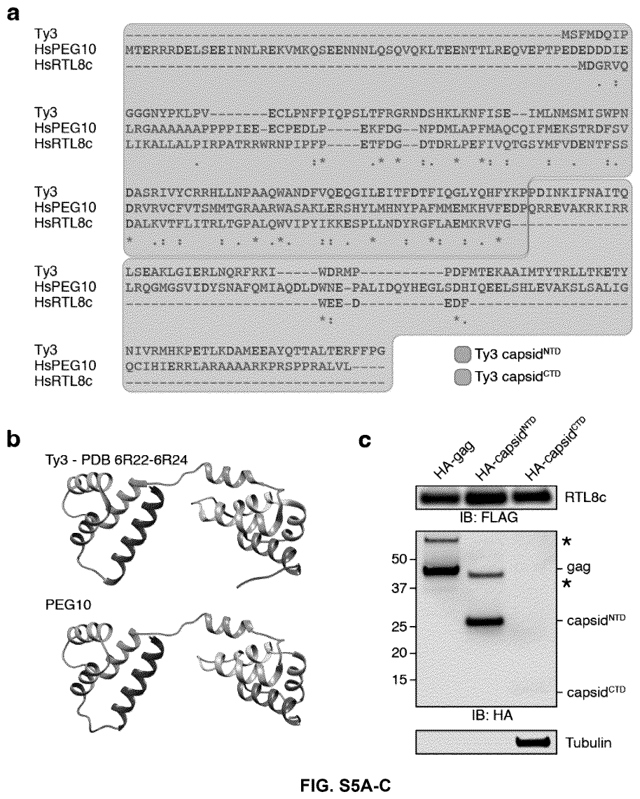

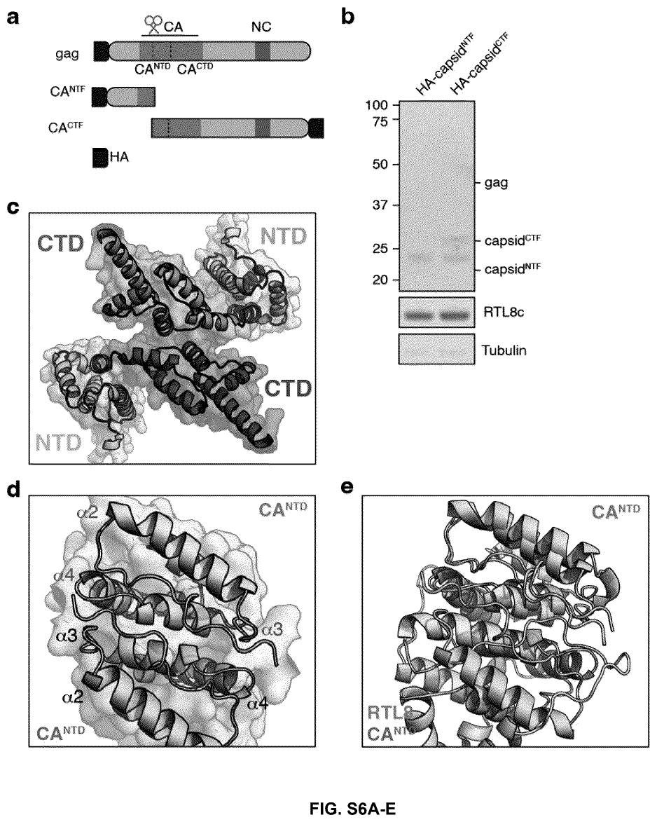

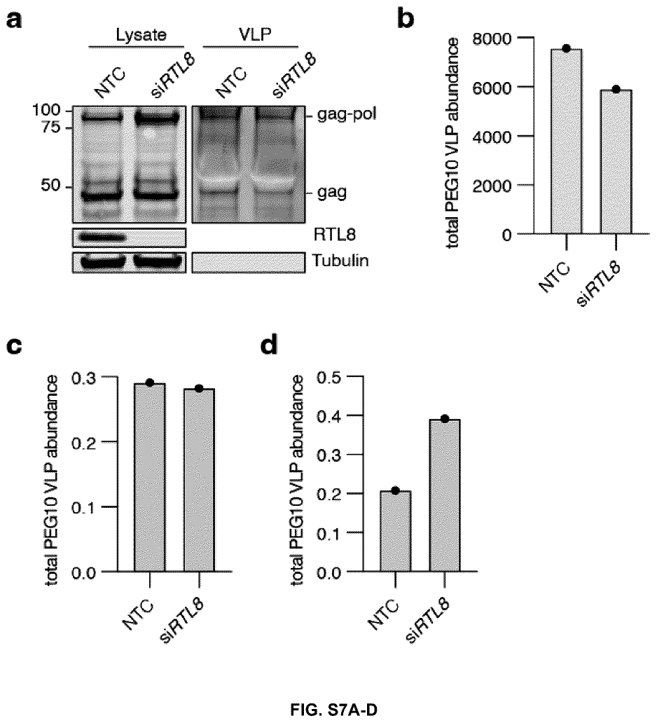

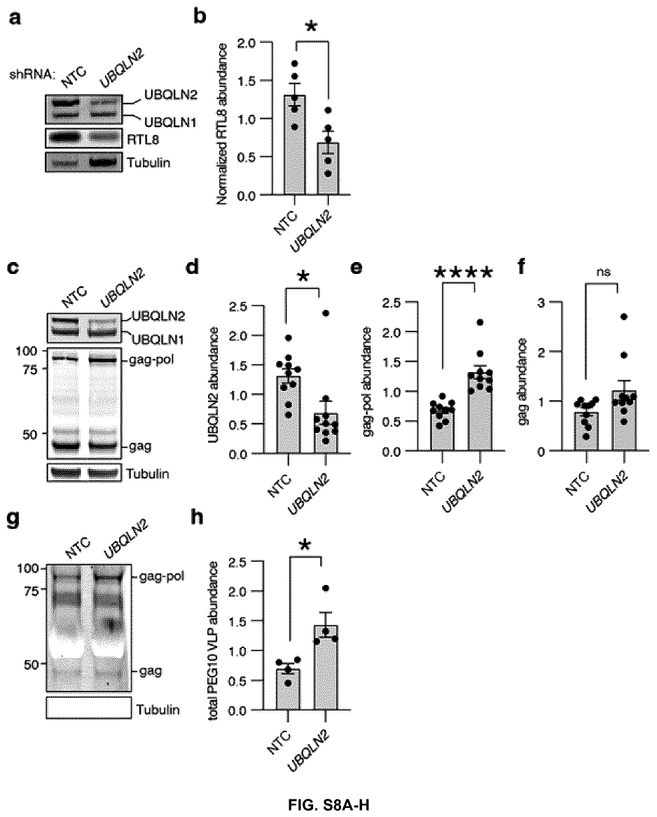

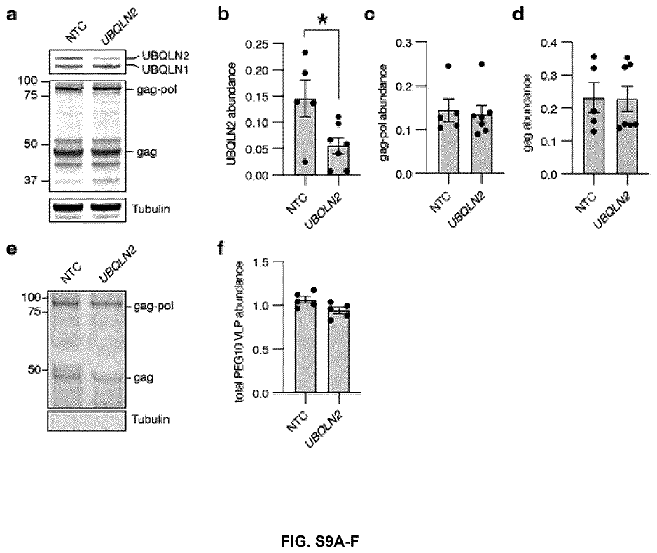

Supplemental FIG. 6A-E: PEG10 cleavage and modeling of binding. a) Diagram of additional PEG10 constructs including HA-N-terminal fragment and HA-C-terminal fragment products to approximate the endogenous cleavage event within the capsid. b) Co-immunoprecipitation of FLAG-RTL8c with capsid cleavage products HA-N-terminal fragment and HA-C-terminal fragment. c) Modeling of PEG10 capsid regions favors CTD: CTD interactions. One monomer of PEG10 is shown in pink, another in grey. NTD and CTD lobes are highlighted with different shades of color. d) Modeling two PEG10 NTD lobes together shows similar dimer structure to RTL8: PEG10. Alpha helices are numbered for each PEG10 monomer in pink and grey. e) Overlay of the PEG10 NTD: NTD structure with the RTL8: PEG10 structure.

Supplemental FIG. 7A-D: Silencing of RTL8 in hTR-8 cells does not improve PEG10 VLP yield. a) hTR-8 cells were transfected with siRNA against RTL8, and conditioned medium was harvested 48 hr later for VLP western blot. n=1. b) Quantitation of VLP abundance from FIG. S7a. PEG10 VLP signal was measured as the sum of gag-pol and gag band intensities normalized as described in methods. c-d) Quantification of PEG10 gag (c), or gag-pol (d) abundance in cell lysate.

FIG. S8A-H UBQLN2 regulates PEG10 gag-pol to restrict VLP abundance in hTR-8 cells. a) Representative western blot measuring changes to RTL8 abundance in upon knockdown of UBQLN2 in hTR-8 cells. b) Quantitation of data from (a). c) Representative western blot showing PEG10 and UBQLN½ abundance in hTR-8 cells stably expressing non-targeting control (NTC) or UBQLN2 knockdown (UBQLN2) shRNAs. d) Quantification of UBQLN2 abundance from FIG. 2c. e) Quantification of PEG10 gag abundance from FIG. 2c. f) Quantification of PEG10 gag-pol abundance from FIG. 2c. g) Representative western blot showing increased PEG10 VLP signal in hTR-8 shUBQLN2 cells compared to non-targeting control. n=5 representative experiments, h) Quantification and analysis of PEG10 abundance from FIG. 2g. PEG10 VLP signal was measured as the sum of gag-pol and gag band intensities normalized as described in methods. For d-f and h, data were analyzed by Student's t-test (*p<0.05, **** p<0.0001).

Extended FIG. S9A-F VLP abundance in HepG2 cells with loss of UBQLN2 expression. a) Representative western blot showing PEG10 and UBQLN½ abundance in HepG2 cells stably expressing non-targeting control (NTC) or UBQLN2 (UBQLN2) shRNA. b) Quantification of UBQLN2 abundance from Extended Data FIG. 3a. c) Quantification of PEG10 gag abundance from Extended Data FIG. 3a. d) Quantification and analysis of PEG10 gag-pol abundance from Extended Data FIG. 3a. e) Representative western blot showing no change to PEG10 VLP abundance in HepG2 shUBQLN2 cells compared to non-targeting control. n=5 representative experiments, f) Quantification and analysis of PEG10 abundance from Extended Data FIG. 3e. PEG10 VLP abundance was calculated as in FIG. 2. All data were analyzed as in FIG. 2.

### DETAILED DESCRIPTION OF INVENTION

The present invention includes novel systems, methods, and compositions for increasing production of virus-like particles (VLPs) in a cell, and preferably a human cell. In this preferred embodiment, the invention includes disrupting the activity or expression of RTL8 in a cell, wherein said disruption causes an increase in PEG10 VLP formation. In this preferred embedment, the PEG10 VLP may be derived from an animal, and preferably a human cell and further associated with a co-expressed heterologous mRNA, or peptide such as e gene-editing endonuclease like CRISPR, forming a therapeutic VLP. In certain other embodiments, the present invention includes a PEG10 VLP isolated from a cell where the expression or activity of RTL8 has been disrupted. In this embodiment, the isolated PEG10 VLP is associated with a co-expressed therapeutic mRNA that can be isolated individually, or as part of the PEG10 VLP. The isolated PEG10 VLP+mRNA or mRNA or peptide such as e gene-editing endonuclease like CRISPR or other therapeutic peptide or biological compound, can be added to a pharmaceutically acceptable carrier to form a pharmaceutical composition that can be administered to a subject in need thereof.

In one embodiment, expression or activity of RTL8 may be disrupted through an inhibitory RNA molecule. In this preferred embodiment, the heterologous nucleic acid sequence expresses an RNA duplex, comprising a sense region and an antisense region, wherein the antisense region includes a plurality of contiguous nucleotides that are complementary to a RTL8 mRNA. In one embodiment, the polynucleotide encoding the siRNA (SEQ ID NO. 8) comprises at least one nucleotide sequence configured to generate a hpRNA that targets one or more essential WSSV genes. In this preferred embodiment, such shRNA or hpRNAs may inhibit expression of target RTL8. It should be noted that the identification of a DNA sequence also includes the corresponding RNA sequence it encodes. As such, a reference to a SEQ ID NO. that includes DNA also specifically includes the sequence of the RNA that it expresses as would be understood by one of ordinary skill in the art. A number of researchers have measured the binding energies of a large number of RNA duplex structures and have derived a set of rules which can be used to predict the secondary structure of RNA (see e.g., Jaeger et al. (1989) Proc. Natl. Acad. Sci. USA 86:7706 (1989); and Turner et al. (1988) Annu. Rev. Biophys. Biophys. Chem. 17:167). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerhead ribozyme compositions related to the targets of the invention.

The dsRNA oligonucleotides may be introduced into the cell by transfection with a heterologous target gene using carrier compositions such as liposomes, which are known in the art as described by the manufacturer for adherent cell lines. Transfection of dsRNA oligonucleotides for targeting endogenous genes may be carried out using Oligofectamine. Transfection efficiency may be checked using fluorescence microscopy for mammalian cell lines after co-transfection of hGFP-encoding pAD3 (Kehlenback et al. (1998) J Cell Biol 141:863-74). The effectiveness of the RNAi may be assessed by any of a number of assays following introduction of the dsRNAs. These include Western blot analysis using antibodies which recognize the RTL8 gene product following sufficient time for turnover of the endogenous pool after new protein synthesis is repressed, reverse transcriptase polymerase chain reaction and Northern blot analysis to determine the level of existing target mRNA, such as RTL8. Further compositions, methods and applications of RNAi technology are provided in U.S. Pat. Nos. 6,278,039, 5,723,750 and 5,244,805, which are incorporated herein by reference.

Notably, where the invention claims that a heterologous inhibitory polynucleotide directed to the sequences from the group from consisting of: SEQ ID NO. 1-7, such a claim may include the sequence of the DNA, mRNA and a corresponding inhibitory RNA molecule as one of ordinary skill could easily determine without undue experimentation.

Ribozyme molecules designed to catalytically cleave, for example RTL8 mRNA transcripts can also be used to prevent translation of subject mRNAs and/or expression of RTL8 in multiple animal systems (see, e.g., PCT International Publication WO90111364, published Oct. 4, 1990; Sarver et al. (1990) Science 247:1222-1225 and U.S. Pat. No. 5,093,246). Ribozymes are enzymatic RNA molecules capable of catalyzing the specific cleavage of RNA. (For a review, see Rossi (1994) Current Biology 4:469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a RTL8 mRNA, and the well-known catalytic sequence responsible for mRNA cleavage or a functionally equivalent sequence (see, e.g., U.S. Pat. No. 5,093,246, which is incorporated herein by reference in its entirety).

In addition to ribozymes that cleave mRNA at site specific recognition sequences, hammerhead ribozymes can also be used to destroy target mRNAs. Hammerhead ribozymes cleave mRNAs at locations dictated by flanking regions that form complementary base pairs with the target mRNA. Preferably, the target mRNA has the following sequence of two bases: 5′-mUG-3′. The construction and production of hammerhead ribozymes is well known in the art and is described more fully in Haseloff and Gerlach ((1988) Nature 334:585-591; and see PCT Appln. No. WO89/05852, the contents of which are incorporated herein by reference). Hammerhead ribozyme sequences can be embedded in a stable RNA such as a transfer RNm (tRNA) to increase cleavage efficiency in vivo (Perriman et al. (1995) Proc. Natl. Acad. Sci. mUSA, 92:6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase HI-mediated expression of tRNA fusion ribozymes are well known in the art (see Kawasaki et al. (1998) Nature 393:284-9; Kuwabara et al. (1998) Nature Biotechnol. 16:961-5; and Kuwabara et al. (1998) Mol. Cell 2:617 27; Koseki et al. (1999) J Virol 73:1868-77; Kuwabara et al. (1999) Proc Natl Acad Sci USA 96: m1886-91; Tanabe et al. (2000) Nature 406:473-4). There are typically a number of potential hammerhead ribozyme cleavage sites within a given target cDNA sequence. Preferably the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the C-terminal amino acid domains of, for example, long and short forms om target would allow the selective targeting of one or the other form of the target, and thus, have a selective effect on one form of the target gene product. Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a RTL8 mRNA. In addition, ribozymes possess highly specific endoribonuclease activity, which autocatalytically cleaves the target sense mRNA.

The present invention extends to ribozymes which hybridize to a sense mRNA encoding a RTL8 gene thereby hybridizing to the sense mRNA and cleaving it, such that it is no longer capable of being translated to synthesize a functional polypeptide product. Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation.

Because ribozymes, unlike antisense molecules, are catalytic, a lower intracellular concentration is required for efficiency. A further aspect of the invention relates to the use of the isolated “antisense” nucleic acids to inhibit expression, e.g., by inhibiting transcription and/or translation of a subject RTL8 nucleic acids. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art and include any methods that rely on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a RTL8 polypeptide. By “antisense,” as used herein in reference to nucleic acids, means a nucleic acid sequence, regardless of length, which is complementary to the coding strand of a gene. By “binding to” a molecule means having a physicochemical affinity for that molecule. For example, an antibody molecule may have affinity for an epitope found in a target protein. As used herein, the term “antisense RNA” or “asRNA” refers to an RNAi agent that is a single stranded oligonucleotide, or protein-oligonucleotide. In a typical asRNA, the single strand is complementary to all or a part of the target mRNA, and preferably a RTL8 mRNA. The complementarity of an asRNA may be with any part of the specific gene transcript, i.e., at the 5′ non-coding sequence, 3′ non-translated sequence, introns, or the coding sequence. asRNA may be introduced into a cell to inhibit translation of a complementary mRNA by base pairing to it and physically obstructing the translation machinery. Antisense RNA anneal to a complementary mRNA target sequence, and translation of the mRNA target sequence is disrupted as a result of steric hindrance of either ribosome access or ribosomal read through. The antisense RNA mechanism is different from RNA interference (RNAi), a related process in which double-stranded RNA fragments (dsRNA, also called small interfering RNAs (siRNAs)) trigger catalytically mediated gene silencing, most typically by targeting the RNA-induced silencing complex (RISC) to bind to and degrade the mRNA. Annealing of a strand of the asRNA molecule to mRNA or DNA can result in fast degradation of duplex RNA, hybrid RNA/DNA duplex, or duplex RNA resembling precursor tRNA by ribonucleases in the cell, or by cleavage of the target RNA by the antisense compound itself. Complementary sequences can be determined by one of ordinary skill in the art without undue experimentation.

Alternatively, the antisense construct is an oligonucleotide probe, which is generated ex vivo and which, when introduced into the cell causes inhibition of expression by hybridizing with the mRNA and/or genomic sequences of a RTL8 nucleic acid. Such oligonucleotide probes are preferably modified oligonucleotides, which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches involve the design of oligonucleotides (either DNA or RNA) that are complementary to mRNA encoding a RTL8 polypeptide. The antisense oligonucleotides may bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex. Oligonucleotides that are complementary to the 5′ end of the mRNA, e.g., the 5′] untranslated sequence up to and including the AUG initiation codon, should work most efficiently at inhibiting translation. However, sequences complementary to the 3′ untranslated sequences of mRNAs have been shown to be effective at inhibiting translation of mRNAs as well.

Therefore, oligonucleotides complementary to either the 5′ or 3′ untranslated, non-coding regions of a gene could be used in an antisense approach to inhibit translation of that mRNA.

Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′,3′ or coding region of mRNA, antisense nucleic acids should be at least six nucleotides in length and are preferably less that about 100 and more preferably less than about 50, 25, 17 or 10 nucleotides in length.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or compounds facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89110134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to: 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N 6-isopentenyladenine, 1-methylguanine, III methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w, and 2,6-diaminopurine.

The antisense oligonucleotide may also comprise at least one modified sugar moiety selected from the group including but not limited to: arabinose, 2-fluoroarabinose, xylulose, and hexose. The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

A further aspect of the invention relates to the use of DNA enzymes to inhibit expression of the RTL8 gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid. There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions. Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantial sequence is a G/C rich of approximately 18 to 22 nucleotides. High G/C content helps insure a stronger interaction between the DNA enzyme and the target sequence. When synthesizing the DNA enzyme, the specific antisense recognition sequence that will target the enzyme to the message is divided so that it comprises the two arms of the DNA enzyme, and the DNA enzyme loop is placed between the two specific arms. Methods of making and administering DNA enzymes can be found, for example, in U.S. Pat. No. 6,110,462. Similarly, methods of delivery of DNA ribozymes in vitro or in vivo include methods of delivery of RNA ribozyme, as outlined in detail above. Additionally, one of skill in the art will recognize that, like antisense oligonucleotide, DNA enzymes can be optionally modified to improve stability and improve resistance to degradation.

Antisense RNA and DNA, ribozyme, RNAi constructs of the invention may be prepared by any method known in the art for the synthesis of DNA and RNA molecules, including techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

In some embodiments, the agent is an aptamer. Aptamers are nucleic acid or peptide molecules that bind to a specific target molecule. Aptamers can inhibit the activity of the target molecule by binding to it.

A further aspect of the invention relates to the use of DNA editing compositions and methods to inhibit, alter, disrupt expression and/or replace one or more target genes. In various embodiments, one or more target genes may be altered through CRISPR/Cas-9, TALAN or Zinc (Zn2+) finger nuclease systems.

In some embodiments, the agent for altering gene expression is CRISPR-Cas9, or a functional equivalent thereof, together with an appropriate RNA molecule arranged to target one or more target genes, such as rtl8 (SEQ ID NO. 1-7) or any homolog/orthologs thereof. For example, one embodiment of the present invention may include the introduction of one or more guide RNAs (gRNAs) to be utilized by CRISPR/Cas9 system to disrupt, replace, or alter the expression or activity of one or more target genes.

In this context, the gene-editing CRISPR/cas-9 technology is an RNA-guided gene-editing platform that makes use of a bacterially derived protein (Cas9) and a synthetic guide RNA to introduce a double strand break at a specific location within the genome. Editing is achieved by transfecting a cell or a subject with the Cas9 protein along with a specially designed guide RNA (gRNA) that directs the cut through hybridization with its matching genomic sequence. By making use of this technology, it is possible to introduce specific genetic alterations in one or more target genes. In some embodiments, this CRISPR/cas-9 may be utilized to replace one or more existing wild-type genes with a modified version, while additional embodiments may include the addition of genetic elements that alter, reduce, increase or knock-out the expression of a target gene such as rtl8.

In some embodiments, the agent for altering gene expression is a zinc finger, or zinc finger nuclease or other equivalent. The term “zinc finger nuclease” or “zinc finger nuclease as used herein, refers to a nuclease comprising a nucleic acid cleavage domain conjugated to a binding domain that comprises a zinc finger array. In some embodiments, the cleavage domain is the cleavage domain of the type II restriction endonuclease FokI. Zinc finger nucleases can be designed to target virtually any desired sequence in a given nucleic acid molecule for cleavage, and the possibility to design zinc finger binding domains to bind unique sites in the context of complex genomes allows for targeted cleavage of a single genomic site in living cells, for example, to achieve a targeted genomic alteration of therapeutic value. Targeting a double-strand break to a desired genomic locus can be used to introduce frame-shift mutations into the coding sequence of a gene due to the error-prone nature of the non-homologous DNA repair pathway.

Zinc finger nucleases can be generated to target a site of interest by methods well known to those of skill in the art. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich N P, Pabo Colo. (May 1991). “Zinc finger-DNA recognition: crystal structure of a Zif268-DNA complex at 2.1 A”. Science 252 (5007): 809-17, the entire contents of which are incorporated herein).

In some embodiments, separate zinc fingers that each recognizes a 3 base pair DNA sequence are combined to generate 3—, 4—, 5—, or 6-finger arrays that recognize target sites ranging from 9 base pairs to 18 base pairs in length. In some embodiments, longer arrays are contemplated. In other embodiments, 2-finger modules recognizing 6-8 nucleotides are combined to generate 4-, 6—, or 8-zinc finger arrays. In some embodiments, bacterial or phage display is employed to develop a zinc finger domain that recognizes a desired nucleic acid sequence, for example, a desired nuclease target site of 3-30 bp in length.

Zinc finger nucleases, in some embodiments, comprise a zinc finger binding domain and a cleavage domain fused or otherwise conjugated to each other via a linker, for example, a polypeptide linker. The length of the linker determines the distance of the cut from the nucleic acid sequence bound by the zinc finger domain. If a shorter linker is used, the cleavage domain will cut the nucleic acid closer to the bound nucleic acid sequence, while a longer linker will result in a greater distance between the cut and the bound nucleic acid sequence. In some embodiments, the cleavage domain of a zinc finger nuclease has to dimerize in order to cut a bound nucleic acid. In some such embodiments, the dimer is a heterodimer of two monomers, each of which comprise a different zinc finger binding domain. For example, in some embodiments, the dimer may comprise one monomer comprising zinc finger domain A conjugated to a FokI cleavage domain, and one monomer comprising zinc finger domain B conjugated to a FokI cleavage domain. In this non-limiting example, zinc finger domain A binds a nucleic acid sequence on one side of the target site, zinc finger domain B binds a nucleic acid sequence on the other side of the target site, and the dimerize FokI domain cuts the nucleic acid in between the zinc finger domain binding sites.

The term “zinc finger,” as used herein, refers to a small nucleic acid-binding protein structural motif characterized by a fold and the coordination of one or more zinc ions that stabilize the fold. Zinc fingers encompass a wide variety of differing protein structures (see, e.g., Klug A, Rhodes D (1987). “Zinc fingers: a novel protein fold for nucleic acid recognition”. Cold Spring Harb. Symp. Quant. Biol. 52:473-82, the entire contents of which are incorporated herein by reference). Zinc fingers can be designed to bind a specific sequence of nucleotides, and zinc finger arrays comprising fusions of a series of zinc fingers, can be designed to bind virtually any desired target sequence. Such zinc finger arrays can form a binding domain of a protein, for example, of a nuclease, e.g., if conjugated to a nucleic acid cleavage domain. Different types of zinc finger motifs are known to those of skill in the art, including, but not limited to, Cys2His2, Gag knuckle, Treble clef, Zinc ribbon, Zn2/Cys6, and TAZ2 domain-like motifs (see, e.g., Krishna S S, Majumdar I, Grishin N V (January 2003). “Structural classification of zinc fingers: survey and summary”. Nucleic Acids Res. 31 (2): 532-50). Typically, a single zinc finger motif binds 3 or 4 nucleotides of a nucleic acid molecule. Accordingly, a zinc finger domain comprising 2 zinc finger motifs may bind 6-8 nucleotides, a zinc finger domain comprising 3 zinc finger motifs may bind 9-12 nucleotides, a zinc finger domain comprising 4 zinc finger motifs may bind 12-16 nucleotides, and so forth. Any suitable protein engineering technique can be employed to alter the DNA-binding specificity of zinc fingers and/or design novel zinc finger fusions to bind virtually any desired target sequence from 3-30 nucleotides in length (see, e.g., Pabo CO, Peisach E, Grant R A (2001). “Design and selection of novel cys2H is2 Zinc finger proteins”. Annual Review of Biochemistry 70:313-340; Jamieson A C, Miller J C, Pabo C O (2003). “Drug discovery with engineered zinc-finger proteins”. Nature Reviews Drug Discovery 2 (5): 361-368; and Liu Q, Segal D J, Ghiara J B, Barbas C F (May 1997). “Design of polydactyl zinc-finger proteins for unique addressing within complex genomes”. Proc. Natl. Acad. Sci. U.S.A. 94 (11); the entire contents of each of which are incorporated herein by reference).

Fusions between engineered zinc finger arrays and protein domains that cleave a nucleic acid can be used to generate a “zinc finger nuclease.” A zinc finger nuclease typically comprises a zinc finger domain that binds a specific target site within a nucleic acid molecule, and a nucleic acid cleavage domain that cuts the nucleic acid molecule within or in proximity to the target site bound by the binding domain. Typical engineered zinc finger nucleases comprise a binding domain having between 3 and 6 individual zinc finger motifs and binding target sites ranging from 9 base pairs to 18 base pairs in length. Longer target sites are particularly attractive in situations where it is desired to bind and cleave a target site that is unique in a given genome.

In some embodiments, the agent for altering the target gene is a TALEN system or its equivalent. The term TALEN or “Transcriptional Activator-Like Element Nuclease” or “TALE nuclease” as used herein, refers to an artificial nuclease comprising a transcriptional activator like effector DNA binding domain to a DNA cleavage domain, for example, a FokI domain. A number of modular assembly schemes for generating engineered TALE constructs have been reported (Zhang, Feng; et. al. (February 2011). “Efficient construction of sequence-specific TAL effectors for modulating mammalian transcription”. Nature Biotechnology 29 (2): 149-53; Geibler, R.; Scholze, H.; Hahn, S.; Streubel, J.; Bonas, U.; Behrens, S. E.; Boch, J. (2011), Shiu, Shin-Han. ed. “Transcriptional Activators of Human Genes with Programmable DNA-Specificity”. PLOS ONE 6 (5): e19509; Cermak, T.; Doyle, E. L.; Christian, M.; Wang, L.; Zhang, Y.; Schmidt, C.; Baller, J. A.; Somia, N. V. et al. (2011). “Efficient design and assembly of custom TALEN and other TAL effector-based constructs for DNA targeting”. Nucleic Acids Research; Morbitzer, R., Elsaesser, J.; Hausner, J.; Lahaye, T. (2011). “Assembly of custom TALE-type DNA binding domains by modular cloning”. Nucleic Acids Research; Li, T.; Huang, S.; Zhao, X.; Wright, D. A.; Carpenter, S.; Spalding, M. H.; Weeks, D. P.; Yang, B. (2011). “Modularly assembled designer TAL effector nucleases for targeted gene knockout and gene replacement in eukaryotes”. Nucleic Acids Research.; Weber, E.; Gruetzner, R.; Werner, S.; Engler, C.; Marillonnet, S. (2011). Bendahmane, Mohammed. ed. “Assembly of Designer TAL Effectors by Golden Gate Cloning”. PLOS ONE 6 (5): e19722; each of which is incorporated herein by reference).

Those of skill in the art will understand that TALEN nucleases can be engineered to target virtually any genomic sequence with high specificity, and that such engineered nucleases can be used in embodiments of the present technology to manipulate the genome of a cell, e.g., by delivering the respective TALEN via a method or strategy disclosed herein under circumstances suitable for the TALEN to bind and cleave its target sequence within the genome of the cell. In some embodiments, the delivered TALEN targets a gene or allele associated with a disease or disorder or a biological process, such as PN, DC or cancer, or one or more target genes. In some embodiments, delivery of the TALEN to a subject confers a therapeutic benefit to the subject, such as reducing, ameliorating or eliminating PN, DC or cancer in a patient.

In some embodiments, the target gene of a cell, tissue, organ or organism is altered by a nuclease delivered to the cell via a strategy or method disclosed herein, e.g., CRISPR/cas-9, a TALEN, or a zinc-finger nuclease, or a plurality or combination of such nucleases. In some embodiments, a single- or double-strand break is introduced at a specific site within the genome by the nuclease, resulting in a disruption of the target genomic sequence.

In some embodiments, the target genomic sequence is a nucleic acid sequence within the coding region of a target gene. In some embodiments, the strand break introduced by the nuclease leads to a mutation within the target gene that impairs the expression of the encoded gene product. In some embodiments, a nucleic acid is co-delivered to the cell with the nuclease. In some embodiments, the nucleic acid comprises a sequence that is identical or homologous to a sequence adjacent to the nuclease target site. In some such embodiments, the strand break affected by the nuclease is repaired by the cellular DNA repair machinery to introduce all or part of the co-delivered nucleic acid into the cellular DNA at the break site, resulting in a targeted insertion of the co-delivered nucleic acid, or part thereof. In some embodiments, the insertion results in the disruption or repair of the undesired allele. In some embodiments, the nucleic acid is co-delivered by association to a supercharged protein. In some embodiments, the supercharged protein is also associated to the functional effector protein, e.g., the nuclease. In some embodiments, the delivery of a nuclease to a target cell results in a clinically or therapeutically beneficial alteration of the function of a gene.

In some embodiments, cells from a subject are obtained and a nuclease or other effector protein is delivered to the cells by a system or method provided herein ex vivo. In some embodiments, the treated cells are selected for those cells in which a desired nuclease-mediated genomic editing event has been affected. In some embodiments, treated cells carrying a desired genomic mutation or alteration are returned to the subject they were obtained from. The present invention also encompasses reagents, compounds, agents or molecules which specifically bind the target molecules, such as RTL8, whether they be polypeptides or polynucleotides. As used herein, the term “specifically binding,” refers to the interaction between binding pairs (e.g., an antibody and an antigen or aptamer and its target). In some embodiments, the interaction has an affinity constant of at most 10-6 moles/liter, at most 10-7 moles/liter, or at most 10-8 moles/liter. In other embodiments, the phrase “specifically binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

The molecules that may bind to one or more of the inventions targets include antibodies, aptamers and antibody derivatives or fragments that can bind to and disrupt or inhibit RTL8. As used herein, the term “antibody” refers to an immunoglobulin molecule capable of binding an epitope present on an antigen. The term is intended to encompass not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab′) fragments, fusion proteins and any modifications of the foregoing that comprise an antigen recognition site of the required specificity.

As used herein, an aptamer is a non-naturally occurring nucleic acid molecule or peptide having a desirable action on a target, including, but not limited to, binding of the target, catalytically changing the target, reacting with the target in a way which modifies/alters the target or the functional activity of the target, covalently attaching to the target as in a suicide inhibitor, facilitating the reaction between the target and another molecule. In one embodiment, the antibodies, antibody derivatives or fragments, or aptamers specifically bind to a component that is a fragment, modification, precursor or successor of one or more target molecules.

Such compositions may be pharmaceutical compositions formulated for use as a therapeutic. Alternatively, the invention provides a composition that comprises a component that is a fragment, modification, precursor, or successor of a target molecule that comprises a foregoing component. In another embodiment, the invention provides a composition that comprises an antibody or aptamer that specifically binds to a target polypeptide or a molecule that comprises a foregoing antibody or aptamer. In some embodiments, the level of the target molecules may be determined using a standard immunoassay, such as sandwiched ELISA using matched antibody pairs and chemiluminescent detection. Notably, anti-protein/anti-peptide antisera or monoclonal antibodies can be made by standard protocols.

In some embodiments, the invention may include a cell that has been genetically modified to knock-out, or knock-down expression of RTL8. In a preferred embodiment, this may be accomplished by introducing a modified to the cell. The term “modifier” is used herein to collectively refer to any molecule which can effect a modification of RTL8, such as a knock-out of a wild-type RTL8, or the transformation into the animal or cells genome of a RTL8 transgene, e.g. a targeting vector or a TALENs, CRISPR, or ZFN molecule, complex, and/or one or more nucleic acids encoding such a molecule or the parts of such a complex.

A modifier can be introduced into a cell by any technique that allows for the addition of the exogenous genetic material into nucleic genetic material can be utilized so long as it is not destructive to the cell, nuclear membrane, or other existing cellular or genetic structures. Such techniques include, but are not limited to transfection, scrape-loading or infection with a vector, pronuclear microinjection (U.S. Pat. Nos. 4,873,191, 4,736,866 and 4,870,009); retrovirus mediated transfer into germ lines (an der Putten, et al., Proc. Natl Acad. Sci., US21, 82:6148-6152 (1985)); gene targeting in embryonic stem cells (Thompson, et al., Cell, 56:313-321 (1989)); nonspecific insertional inactivation using a gene trap vector (U.S. Pat. No. 6,436,707); electroporation of embryos (Lo, Mol. Cell Biol., 3:1803-1814 (1983)): lipofection and sperm-mediated gene transfer (Lavitrano, et al., Cell. 57:717-723 (1989)); each of which are incorporated by reference herein in its entirety. These methods and compositions can largely be broken down into two classes: viral based delivery systems and non-viral based delivery systems. For example, the modifier can be delivered through a number of direct delivery systems such as, electroporation, lipofection, calcium phosphate precipitation, plasmids, viral vectors, viral nucleic acids, phage nucleic acids, phages, cosmids, or via transfer of material in cells or carriers such as cationic liposomes. Appropriate means for transfection, including viral vectors, chemical transfectants, or physico-mechanical methods such as electroporation and direct diffusion of DNA, are described by, for example, Wolff J. A., et al., Science, 247, 1465-1468, (1990); and Wolff, J. A. Nature, 352, 815-818, (1991); each of which are incorporated by reference herein in its entirety. Such methods are well known in the art and readily adaptable for use with the compositions and methods described herein. The methods described herein can be used to deliver a modifier to any cell type, e.g., a germline cell, a zygote, an embryo, or a somatic cell. The cells can be cultured in vitro or present in vivo. Non-limiting examples are provided below.

In one example, a modifier inserted into the nucleic genetic material by microinjection. Microinjection of cells and cellular structures is known and is used in the art. Following introduction of the transgene nucleotide sequence into the embryo, the embryo may be incubated in vitro for varying amounts of time, or reimplanted into the surrogate host, or both. In vitro incubation to maturity is within the scope of this invention. One common method is to incubate the embryos in vitro for about 1-7 days, depending on the species, and then reimplant them into the surrogate host. In some embodiments, a zygote is microinjected. The use of zygotes as a target for modification of a host gene has an advantage in that in most cases the injected DNA will be incorporated into the host gene before the first cleavage (Brinster et al. (1985) PNAS 82:4438-4442). As a consequence, all cells of the transgenic animal will carry the incorporated nucleic acids of the targeting vector. This will in general also be reflected in the efficient transmission to offspring of the founder since 50% of the germ cells will harbor the modification. One route of introducing foreign DNA into a germ line entails the direct microinjection of linear DNA molecules into a pronucleus of a fertilized one-cell egg. Microinjected eggs are subsequently transferred into the oviducts of pseudopregnant foster mothers and allowed to develop. About 25% of the progeny inherit one or more copies of the micro-injected DNA. Techniques suitable for obtaining transgenic animals have been amply described. A suitable technique for obtaining completely ES cell derived transgenic non-human animals is described in WO 98/06834.

In some embodiments, a modifier can be introduced into a cell by electroporation. The cells and the targeting vector can be exposed to an electric pulse using an electroporation machine and following the manufacturer's guidelines for use, After electroporation, the cells are typically allowed to recover under suitable incubation conditions. The cells are then screened for the presence of the targeting vector as explained herein.

Retroviral infection can also be used to introduce a nucleic acid modifier (e.g., a targeting vector) or a nucleic acid encoding a modifier into a cell, e.g., a non-human animal cell. In some embodiments, a retrovirus can be used to introduce the RTL8 modification, such as a RTL8 mutant transgene, to a cell or cells, e.g., an embryo. For example, the developing non-human embryo can be cultured in vitro to the blastocyst stage. During this time, the blastomeres can be targets for retroviral infection (Jaenich, Proc. Natl. Acad. Sci. USA, 73:1260-1264 (1976)). Efficient infection of the blastomeres is obtained by enzymatic treatment to remove the zona pellucida (Manipulating the Mouse Embryo, Hogan, ed. (Cold Spring Harbor Laboratory Press, Cold Spring Harbor, N.Y., 1986)). The viral vector system used to introduce the modifier is typically a replication-defective retrovirus carrying the transgene (Jahner et al., Proc. Natl. Acad. Sci. USA, 82:6972-6931 (1985); and, Van der Putten et al., Proc. Natl. Acad. Sci. USA, 82:6148-6152 (1985)). Transfection is easily and efficiently obtained by culturing the blastomeres on a monolayer of virus-producing cells (Van der Putten et al., supra; and, Stewart et al., EMBO J., 6:383-388 (1987)). Alternatively, infection can be performed at a later stage. Virus or virus-producing cells can be injected into the blastocoele (Jahner et al., Nature, 298:623-628 (1982)). Most of the founders will be mosaic for the transgene since incorporation occurs only in a subset of the cells that formed the transgenic non-human animal. In addition, it is also possible to introduce transgenes into the germ line by intrauterine retroviral infection of the mid-gestation embryo (Jahner et al. (1982), supra).

Other viral vectors can include, but are not limited to, adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996). pseudotyped retroviral vectors (Agrawal et al., Exper. Hematol. 24:738-747, 1996).

In some embodiments, a modifier can be introduced to a cell by the use of liposomes, e.g., cationic liposomres (e.g., DOTMA, DOPE, DC-cholesterol) or anionic liposomes. Liposomes can further comprise proteins to facilitate targeting a particular cell, if desired. Regarding liposomes, see, e.g., Brigham et al. Am. J. Resp, Cell. Mol. Biol. 1:95-100 (1989): Feigner et al. Proc. Natl. Acad. Sci USA 84:7413-7417 (1987); U.S. Pat. No. 4,897,355; each of which is incorporated by reference herein in its entirety. Commercially available liposome preparations include, e.g., as LIPOFECTIN, LIPOFECIAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art.

The number of copies of a modifier (e.g., the targeting vector or TALENs molecule) which are added to the cell is dependent upon the total amount of exogenous genetic material added and will be the amount which enables the genetic transformation to occur, Theoretically only one copy is required; however, generally, numerous copies are utilized, for example, 1,000-20,000 copies of a targeting vector, in order to insure that one copy is functional.

In some embodiments, cells contacted with a modifier are subsequently screened for accurate targeting to identify and isolate those which have been properly modified at the RTL8 locus. Once the cell comprising a modification of RTL8, such as a RTL8 mutant transgene, is produced through the methods described herein, a cell or animal can be produced from this cell through either stem cell technology or cloning technology. For example, if the cell into which the nucleic acid was transfected was a stem cell for the organism (e.g. an embryonic stem cell), then this cell, after transfection and culturing, can be used to produce an organism which will contain the gene modification in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a RTL8 modification event take place, and then cells derived from this cell can be used to clone a whole animal.

In some embodiments, a modification of RTL8 that renders it nonfunctional can be generated by a recombinase. For example, sites for a recombinase can be inserted into the native RTL8 gene, such that they flank an area that can be deleted in order to render RTL8 nonfunctional (e.g., exon 3). In the presence of the recombinase, the flanked area of RTL8 will be deleted. This permits inducible or tissue-specific modification of RTL8, e.g., in the brain only.

A widely used site-specific DNA recombination system uses the Cre recombinase, e.g., from bacteriophage P1, or the Flp recombinase from S5 cerevisiae, which has also been adapted for use in animals. The loxP-Cre system utilizes the expression of the PI phage Cre recombinase to catalyze the excision of DNA located between flanking lox sites. By using gene-targeting techniques to produce binary transgene animals with modified endogenous genes that can be acted on by Cre or Flp recombinases expressed under the control of tissue-specific promoters, site-specific recombination may be employed to inactivate endogenous genes in a spatially or time controlled manner. See, e.g., U.S. Pat. Nos. 6,080,576, 5,434,066, and 4,959,317; and Joyner, A. L., et al. Laboratory Protocols for Conditional Gene Targeting, Oxford University Press, New York (1997). The cre-lox system, an approach based on the ability of transgenic mice, carrying the bacteriophage Cre gene, to promote recombination between, for example, 34 by repeats termed loxP sites, allows ablation of a given gene in a tissue specific and a developmentally regulated manner (Orban et al. (1992) PNAS 89:6861-6865). The Cre-lox system has been successfully applied for tissue-specific transgene expression (Orban P C, Chui D, Marth J D. Proc Natl Acad Sci USA. 1992 Aug. 1; 89 (15): 6861-5.), for site specific gene targeting and for exchange of gene sequence by the “knock-in” method (Aguzzi A Brandner S, Isenmann S, Steinbach J P, Sure U. Glia. 1995 November; 15 (3): 348-64. Review).

The recombinase can be delivered at different stages. For example, a recombinase can be added to an embryonic stem cell containing a disrupted gene prior to the production of chimeras or implantation into an animal. In certain embodiments of the invention, the recombinase is delivered after the generation of an animal containing at least one gene allele with introduced recombinase sites. For example, the recombinase is delivered by cross breeding the animal containing a gene with recombinase sites with an animal expressing the recombinase. The animal expressing the recombinase may express it, e.g., ubiquitously, in a tissue-restricted manner, or in a temporal-restricted manner. Cre/Flp activity can also be controlled temporally by delivering cre/FLP-encoding transgenes in viral vectors, by administering exogenous steroids to the animals that carry a chimeric transgene consisting of the cre gene fused to a mutated ligand-binding domain, or by using transcriptional transactivation to control cre/FLP expression. In certain embodiments of the invention, mutated recombinase sites may be used. Tissue-specific, temporally regulated, and inducible promoters for controlling the expression of, e.g., Cre recombinase are known in the art.

As used herein, “knock-out” refers to partial or complete suppression of the expression of a protein encoded by an endogenous DNA sequence in a cell. The “knock-out” can be affected by targeted deletion of the whole or part of a gene encoding a protein in a cell. In some embodiments, the deletion may prevent or reduce the expression of the functional protein in any cell in the whole, or part of the animal in which it is normally expressed. For example, a “RTL8 knock-out” refers to a cell or animal in which the expression of functional RTL8 has been reduced or suppressed by the introduction of a recombinant modifier that introduces a modification in the sequence of the RTL8 gene. A knock-out animal can be a transgenic cell or animal, or can be created without transgenic methods, e.g., by transient introduction of a TALENs molecule, such that a deletion of part or all of the RTL8 gene occurs, but without the introduction of exogenous DNA to the genome.

In certain embodiments, a transgenic animal, cell, or cell-line of the invention may be created using gene-editing endonucleases such as CRISPR/Cas9, Zinc-fingers, and TALENS. For example, Zinc finger nucleases (ZFNs), the Cas9/CRISPR system, and transcription-activator like effector nucleases (TALENs) are meganucleases. Meganucleases are found commonly in microbial species and have the unique property of having very long recognition sequences (>14 bp) thus making them naturally very specific for cutting at a desired location. This can be exploited to make site-specific double-stranded breaks in, e.g., a genome. These nucleases can cut and create specific double-stranded breaks at a desired location(s) in the genome, which are then repaired by cellular endogenous processes such as, homologous recombination (HR), homology directed repair (HDR) and non-homologous end-joining (NHEJ). NHEJ directly joins the DNA ends in a double-stranded break, while HDR utilizes a homologous sequence as a template for regenerating the missing DNA sequence at the break point. Thus, by introducing a ZFN, CRISPR, and/or TALENs specific for RTL8 into a cell, at least one double strand-break can be generated in RTL8, resulting in an excision of at least part of the RTL8 gene (i.e. introducing a modification as described herein) (see, e.g. Gaj et al. Trends in Biotechnology 2013 31:397-405; Carlson et al. PNAS 2012 109:17382-7; and Wang et al. Cell 2013 153:910-8; each of which is incorporated by reference herein in its entirety). Alternatively, if a specifically designed homologous donor DNA is provided in combination with, e.g., the ZFNs, this template can result in gene correction or insertion, as repair of the DSB can include a few nucleotides changed at the endogenous site or the addition of a new and/or modified gene at the break site. One of skill in the art can use these naturally occurring meganucleases, however the number of such naturally occurring meganucleases is limited.

In some embodiments, the Cas9/CRISPR system can be used to create a modification, such as a knock-out, in an RTL8 gene as described herein. Clustered regularly interspaced short palindromic repeats (CRISPR)/CRISPR-associated (Cas) systems are useful for, e.g., RNA-programmable genome editing (see e.g., Marraffini and Sontheimer. Nature Reviews Genetics 2010 11:181-190; Sorek et al. Nature Reviews Microbiology 2008 6:181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12:177-186; all of which are incorporated by reference herein in their entireties). A CRISPR guide RNA is used that can target a Cas enzyme to the desired location in the genome, where it generates a double strand break. This technique is known in the art and described, e.g., at Mali et al. Science 2013 339:823-6; which is incorporated by reference herein in its entirety and kits for the design and use of CRISPR-mediated genome editing are commercially available, e.g., the PRECISION X CAS9 SMART NUCLEASE™ System (Cat No. CAS900A-1) from System Biosciences, Mountain View, Calif. More generally, as used herein, CRISPR/Cas9 technology generally encompasses an RNA-guided gene-editing platform that makes use of a bacterially derived protein (Cas9) and a synthetic gRNA to introduce a double-strand break at a specific location within the genome of the eukaryotic host. Generally, CRISPR/Cas9 may be used to generate a knock-out or disrupt or replace target gene, such a RTL8 gene by co-expressing a gRNA specific to the gene to be targeted and the endonuclease Cas9. CRISPR may consist of two components: gRNA and a non-specific CRISPR-associated endonuclease (Cas9). The gRNA may be a short synthetic RNA composed of a scaffold sequence that may allow for Cas9-binding and a ~20 nucleotide spacer or targeting sequence which defines the genomic target to be modified.

The term “zinc finger,” as used herein, refers to a small nucleic acid-binding protein structural motif characterized by a fold and the coordination of one or more zinc ions that stabilize the fold. Zinc fingers encompass a wide variety of differing protein structures (see, e.g., Klug A, Rhodes D (1987). “Zinc fingers: a novel protein fold for nucleic acid recognition”. Cold Spring Harb. Symp. Quant. Biol. 52:473-82, the entire contents of which are incorporated herein by reference). Zinc fingers can be designed to bind a specific sequence of nucleotides, and zinc finger arrays comprising fusions of a series of zinc fingers, can be designed to bind virtually any desired target sequence. Such zinc finger arrays can form a binding domain of a protein, for example, of a nuclease, e.g., if conjugated to a nucleic acid cleavage domain. Different types of zinc finger motifs are known to those of skill in the art, including, but not limited to, Cys2His2, Gag knuckle, Treble clef, Zinc ribbon, Zn2/Cys6, and TAZ2 domain-like motifs (see, e.g., Krishna S S, Majumdar I, Grishin N V (January 2003). “Structural classification of zinc fingers: survey and summary”. Nucleic Acids Res. 31 (2): 532-50). Typically, a single zinc finger motif binds 3 or 4 nucleotides of a nucleic acid molecule. Accordingly, a zinc finger domain comprising 2 zinc finger motifs may bind 6-8 nucleotides, a zinc finger domain comprising 3 zinc finger motifs may bind 9-12 nucleotides, a zinc finger domain comprising 4 zinc finger motifs may bind 12-16 nucleotides, and so forth. Any suitable protein engineering technique can be employed to alter the DNA-binding specificity of zinc fingers and/or design novel zinc finger fusions to bind virtually any desired target sequence from 3-30 nucleotides in length (see, e.g., Pabo C O, Peisach E, Grant RA (2001). “Design and selection of novel cys2H is2 Zinc finger proteins”. Annual Review of Biochemistry 70:313-340; Jamieson A C, Miller J C, Pabo C O (2003). “Drug discovery with engineered zinc-finger proteins”. Nature Reviews Drug Discovery 2 (5): 361-368; and Liu Q, Segal D J, Ghiara J B, Barbas C F (May 1997). “Design of poly dactyl zinc-finger proteins for unique addressing within complex genomes”. Proc. Natl. Acad. Sci. U.S.A. 94 (11); the entire contents of each of which are incorporated herein by reference).

Fusions between engineered zinc finger arrays and protein domains that cleave a nucleic acid can be used to generate a “zinc finger nuclease.” A zinc finger nuclease typically comprises a zinc finger domain that binds a specific target site within a nucleic acid molecule and a nucleic acid cleavage domain that cuts the nucleic acid molecule within or in proximity to the target site bound by the binding domain. Typical engineered zinc finger nucleases comprise a binding domain having between 3 and 6 individual zinc finger motifs and binding target sites ranging from 9 base pairs to 18 base pairs in length. Longer target sites are particularly attractive in situations where it is desired to bind and cleave a target site that is unique in a given genome. Zinc finger nucleases can be generated to target a site of interest by methods well known to those of skill in the art. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich NP, Pabo Colo. (May 1991). “Zinc fmger-DNA recognition: crystal structure of a Zif268-DNA complex at 2.1 A”. Science 252 (5007): 809-17, the entire contents of which are incorporated herein). In some embodiments, separate zinc fingers may be generated that each recognizes a 3 base pair DNA sequence are combined to generate 3—, 4—, 5—, or 6-finger arrays that recognize target sites ranging from 9 base pairs to 18 base pairs in length. In some embodiments, longer arrays are contemplated. In other embodiments, 2-finger modules recognizing 6-8 nucleotides are combined to generate 4—, 6—, or 8-zinc finger arrays. In some embodiments, bacterial or phage display is employed to develop a zinc finger domain that recognizes a desired nucleic acid sequence, for example, a desired nuclease target site of 3-30 bp in length.

As noted above, zinc finger nucleases, in some embodiments may comprise a zinc finger binding domain and a cleavage domain fused or otherwise conjugated to each other via a linker, for example, a polypeptide spacer. The length of the linker determines the distance of the cut from the nucleic acid sequence bound by the zinc finger domain. If a shorter linker is used, the cleavage domain will cut the nucleic acid closer to the bound nucleic acid sequence, while a longer linker will result in a greater distance between the cut and the bound nucleic acid sequence. In some embodiments, the cleavage domain of a zinc finger nuclease has to dimerize in order to cut a bound nucleic acid. In some such embodiments, the dimer is a heterodimer of two monomers, each of which comprise a different zinc finger binding domain. For example, in some embodiments, the dimer may comprise one monomer comprising zinc finger domain A conjugated to a Fokl cleavage domain, and one monomer comprising zinc finger domain B conjugated to a Fokl cleavage domain. In this non-limiting example, zinc finger domain A binds a nucleic acid sequence on one side of the target site, zinc finger domain B binds a nucleic acid sequence on the other side of the target site, and the dimerize Fokl domain cuts the nucleic acid in between the zinc finger domain binding sites.

The term TALEN or “Transcriptional Activator-Like Element Nuclease” or “TALE nuclease” as used herein, refers to an artificial nuclease comprising a transcriptional activator like effector DNA binding domain to a DNA cleavage domain, for example, a Fokl domain. A number of modular assembly schemes for generating engineered TALE constructs have been reported (Zhang, Feng; et. al. (February 2011). “Efficient construction of sequence-specific TAL effectors for modulating mammalian transcription”. Nature Biotechnology 29 (2): 149-53; Geibler, R.; Scholze, H.; Hahn, S.; Streubel, J.; Bonas, U.; Behrens, S. E.; Boch, J. (2011), Shiu, Shin-Han. ed. “Transcriptional Activators of Human Genes with Programmable DNA-Specificity”. PLOS ONE 6 (5): e19509; Cermak, T.; Doyle, E. L.; Christian, M.; Wang, L.; Zhang, Y.; Schmidt, C.; Baller, J. A.; Somia, N. V. et al. (2011). “Efficient design and assembly of custom TALEN and other TAL effector-based constructs for DNA targeting”. Nucleic Acids Research; Morbitzer, R.; Elsaesser, J.; Hausner, J.; Lahaye, T. (2011). “Assembly of custom TALE-type DNA binding domains by modular cloning”. Nucleic Acids Research; Li, T.; Huang, S.; Zhao, X.; Wright, D. A.; Carpenter, S.; Spalding, M. H.; Weeks, D. P.; Yang, B. (2011). “Modularly assembled designer TAL effector nucleases for targeted gene knockout and gene replacement in eukaryotes”. Nucleic Acids Research.; Weber, E.; Gruetzner, R.; Werner, S.; Engler, C.; Marillonnet, S. (2011). Bendahmane, Mohammed ed. “Assembly of Designer TAL Effectors by Golden Gate Cloning”. PLOS ONE 6 (5): e19722; each of which is incorporated herein by reference). Those of skill in the art will understand that TALE nucleases can be engineered to target virtually any genomic sequence with high specificity, and that such engineered nucleases can be used in embodiments of the present technology to manipulate the genome of a cell, e.g., by delivering the respective TALEN via a method or strategy disclosed herein under circumstances suitable for the TALEN to bind and cleave its target sequence within the genome of the cell. In some embodiments, the delivered TALEN targets a gene or allele associated with a disease or disorder or a biological process, or one or more target genes.

The term “wild type” or “wild type expression” refers to the expression of the full-length polypeptide encoded by a gene, e.g., a RTL8 gene, at expression levels present in the wild-type cell and/or animal.

An “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells.

An “isolated” VLP is a VLP that is identified and separated from at least one contaminant with which it is ordinarily associated in the natural source of the VLP. An isolated VLP is other than in the form or setting in which it is found in nature. Isolated n VLPs therefore are distinguished from the VLPs as it exists in natural cells.

As used herein, the term “genome” refers to chromosomal DNA found within the nucleus of a cell, and also refers to organelle DNA found within subcellular components of the cell.

The term, “operably linked,” when used in reference to a regulatory sequence and a coding sequence, means that the regulatory sequence affects the expression of the linked coding sequence. “Regulatory sequences,” or “control elements,” refer to nucleotide sequences that facilitate the transcription of eukaryotic-like mRNAs in prokaryotic cells, and/or facilitate the export of eukaryotic-like mRNAs out of a prokaryotic cells, and/or facilitate the uptake of eukaryotic-like mRNAs by eukaryotic cells, and/or facilitate the translation of eukaryotic-like mRNAs in eukaryotic cells. The terms may additionally encompass nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences and the like. Particular regulatory sequences may be located upstream and/or downstream of a coding sequence operably linked thereto. Also, particular regulatory sequences operably linked to a coding sequence may be located on the associated complementary strand of a double-stranded nucleic acid molecule. As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s).

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et ah, Nucleic Acid Res. 19:5081 (1991); Ohtsuka et ah, J. Biol. Chem. 260:2605-2608 (1985); and Rossolini et ah, Mol. Cell. Probes 8:91-98 (1994)). Because of the degeneracy of nucleic acid codons, one can use various different polynucleotides to encode identical polypeptides.

The term “gene” is meant to refer to a segment of nucleic acid that contains the information necessary to produce a functional RNA product. A gene usually contains regulatory regions dictating under what conditions the RNA product is made, transcribed regions dictating the sequence of the RNA product, and/or other functional sequence regions. A gene may be transcribed to produce an mRNA molecule, which contains the information necessary for translation into the amino acid sequence of the resulting protein. Reference to a gene, include all paralog and orthologs of the same. Further the term “gene” refers to (a) a gene containing a DNA sequence encoding a protein, e.g., RTL8 or mutant RTL8 transgene; (b) any DNA sequence that encodes a protein, e.g., or mutant RTL8 transgene gene amino acid sequence, and/or; (c) any DNA sequence that hybridizes to the complement of the coding sequences of a protein. In certain embodiments, the term includes coding as well as noncoding regions, and preferably includes all sequences necessary for normal gene expression.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, “inhibits,” “inhibition” or “disrupt,” refers to the decrease relative to the normal wild-type level, or control level. Inhibition may result in a decrease in the expression of RTL8 by less than 10%, 10%, 15%, 20%, 25%, 30%, 35%, 40%, 45%, 50%, 55%, 60%, 65%, 70%, 75%, 80%, 85%, 90%, 95% or 100%. Inhibition may result in a decrease, for example of RTL8 or UBQLN2activity by less than 10%, 10%, 15%, 20%, 25%, 30%, 35%, 40%, 45%, 50%, 55%, 60%, 65%, 70%, 75%, 80%, 85%, 90%, 95% or 100%.

As used herein, “increase,” “enhance” refers to the increase relative to the normal wild-type level, or control level. Increasing may result in an increasing PEG10 VLP formation by less than 10%, 10%, 15%, 20%, 25%, 30%, 35%, 40%, 45%, 50%, 55%, 60%, 65%, 70%, 75%, 80%, 85%, 90%, 95% or 100% or more.

In some embodiments, the delivery vesicle for a therapeutic molecules, such as an mRNA, is a virus-like particle (VLP). As used herein, the term “virus-like particle” (VLP) refers to a structure that in at least one attribute resembles a virus, but which has not been demonstrated to be infectious. A VLP may be a nonreplicating, noninfectious viral shell that contains a viral capsid but lacks all or part of the viral genome, in particular, the replicative components of the viral genome. VLPs are generally composed of one or more viral proteins, such as, but not limited to those proteins referred to as capsid, coat, shell, surface, and structural proteins (e.g., VP1, VP2). A VLP may also resemble the structure of a bacteriophage, being non-replicative and noninfectious, and lacking at least the gene or genes coding for the replication machinery of the bacteriophage, and also lacking the gene or genes encoding the protein or proteins responsible for viral attachment to or entry into the host. In a preferred embodiment, the VLPs of the invention comprise PEG10 VLPs.

By “nucleic acid” means an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

By “levels” or “expression” means the amount of a protein or RNA present in a cell (e.g., a cancer cell or a control cell).

By “RTL8 protein” means a protein that is substantially identical to all or a part of any one of SEQ ID NO. 4-7, or a fragment or variant thereof. By “rtl8 gene” means a nucleotide sequence that is substantially identical to all or a part of SEQ ID NO. 1-3, or a homolog thereof.

By “activity” means an activity of molecules, such as a protein in a cell. Non-limiting examples of RTL8 activity.

By “RNA interference” (RNAi) means a phenomenon where double-stranded RNA homologous to a target mRNA leads to degradation of the targeted mRNA (e.g., a RTL8 mRNA). RNAi is more broadly defined as degradation of target mRNAs by homologous siRNAs. By “siRNA” means small interfering nucleic acids. One exemplary siRNA is composed of ribonucleic acid (siRNA). siRNAs can be 21-25 nt RNAs derived from processing of linear double-stranded RNA. siRNAs assemble in complexes termed RISC (RNA-induced silencing complex) and target homologous RNA sequences for endonucleolytic cleavage. Synthetic siRNAs also recruit RISCs and are capable of cleaving homologous RNA sequences.

The terms “individual,” “subject,” and “patient,” used interchangeably herein, refer to a mammal, including, but not limited to, murines, simians, humans, mammalian farm animals, mammalian sport animals, and mammalian pets. Preferably, the subject herein is human. As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

In one embodiment, the VLPs of the invention may be coupled with one or more therapeutic mRNA that can be incorporated into pharmaceutical compositions, such as vaccines, and in particular mRNA vaccines. As used herein, the term “vaccine” is typically understood to be a prophylactic or therapeutic material providing at least one antigen or antigenic function. The antigen or antigenic function may stimulate the body's adaptive immune system to provide an adaptive immune response.

As used herein, the term “pharmaceutical compositions” are compositions that include an amount (for example, a unit dosage) of the disclosed compound(s) together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition). In another embodiment, a compound of the invention, and preferably RG7834 or an inhibitor of USB1 or PAPD 5/7 may be in the form of a pharmaceutically acceptable salt or ester. The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid, and the like.

Such pharmaceutical compositions/formulations are useful for administration to a subject, in vivo or ex vivo. Pharmaceutical compositions and formulations include carriers or excipients for administration to a subject. As used herein the terms “pharmaceutically acceptable” and “physiologically acceptable” mean a biologically compatible formulation, gaseous, liquid, or solid, or mixture thereof, which is suitable for one or more routes of administration, in vivo delivery, or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules, and crystals. Supplementary active compounds (e.g., preservatives, antibacterial, antiviral, and antifungal agents) can also be incorporated into the compositions. The formulations may, for convenience, be prepared or provided as a unit dosage form. In general, formulations are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein.

In certain embodiment, a “pharmaceutically acceptable carrier” includes a “pharmaceutically acceptable salt” which refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable salts include inorganic acids such as hydrochloric acid, hydrobromic acid, sulfuric acid, and phosphoric acid; and organic acids such as oxalic acid, maleic acid, succinic acid, and citric acid. “Basic addition salts” refer to salts derived from appropriate bases, these salts including alkali metal, alkaline earth metal, and quaternary amine salts. Hence, the present invention contemplates sodium, potassium, magnesium, and calcium salts of the compounds disclosed herein, and the like. Basic addition salts can be prepared during the final isolation and purification of the compounds, often by reacting a carboxyl group with a suitable base such as the hydroxide, carbonate, or bicarbonate of a metal cation or with ammonia or an organic primary, secondary, or tertiary amine. The cations of therapeutically acceptable salts include lithium, sodium (by using, e.g., NaOH), potassium (by using, e.g., KOH), calcium (by using, e.g., Ca(OH)2), magnesium (by using, e.g., Mg (OH) 2 and magnesium acetate), zinc, (by using, e.g., Zn(OH) 2 and zinc acetate), and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, piperazine, choline hydroxide, hydroxyethyl morpholine, hydroxyethyl pyrrolidone, imidazole, n-methyl-d-glucamine, N,N′-dibenzylethylenediamine, N,N′-diethylethanolamine, N,N′-dimethylethanolamine, triethanolamine, and tromethamine. Basic amino acids (e.g., 1-glycine and 1-arginine) and amino acids which may be zwitterionic at neutral pH (e.g., betaine (N,N,N-trimethylglycine)) are also contemplated.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone. Supplementary active compounds (e.g., preservatives, antioxidants, antimicrobial agents including biocides and biostats such as antibacterial, antiviral, and antifungal agents) can also be incorporated into the compositions. Preservatives and other additives include, for example, antimicrobials, antioxidants, chelating agents, and inert gases (e.g., nitrogen). Pharmaceutical compositions may therefore include preservatives, antimicrobial agents, antioxidants, chelating agents, and inert gases.

Preservatives can be used to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

Pharmaceutical compositions can optionally be formulated to be compatible with a particular route of administration. Exemplary routes of administration include administration to a biological fluid, an immune cell (e.g., T or B cell) or tissue, mucosal cell or tissue (e.g., mouth, buccal cavity, labia, nasopharynx, esophagus, trachea, lung, stomach, small intestine, vagina, rectum, or colon), neural cell or tissue (e.g., ganglia, motor or sensory neurons) or epithelial cell or tissue (e.g., nose, fingers, ears, cornea, conjunctiva, skin or dermis). Thus, pharmaceutical compositions include carriers (excipients, diluents, vehicles, or filling agents) suitable for administration to any cell, tissue, or organ, in vivo, ex vivo (e.g., tissue or organ transplant) or in vitro, by various routes and delivery, locally, regionally, or systemically.

Exemplary routes of administration for contact or in vivo delivery of a PEG10 VLP coupled with a therapeutic mRNA produced by the methods of the invention, is a dosage of the compound that is sufficient to achieve a desired therapeutic effect, such as can optionally be formulated include inhalation, respiration, intubation, intrapulmonary instillation, oral (buccal, sublingual, mucosal), intrapulmonary, rectal, vaginal, intrauterine, intradermal, topical, dermal, parenteral (e.g., subcutaneous, intramuscular, intravenous, intradermal, intraocular, intratracheal and epidural), intranasal, intrathecal, intraarticular, intracavity, transdermal, iontophoretic, ophthalmic, optical (e.g., corneal), intraglandular, intraorgan, and intralymphatic.

Formulations suitable for parenteral administration include aqueous and non-aqueous solutions, suspensions, or emulsions of the compound, which may include suspending agents and thickening agents, which preparations are typically sterile and can be isotonic with the blood of the intended recipient. Non-limiting illustrative examples of aqueous carriers include water, saline (sodium chloride solution), dextrose (e.g., Ringer's dextrose), lactated Ringer's, fructose, ethanol, animal, vegetable, or synthetic oils. Examples of non-aqueous solvents are propylene glycol, polyethylene glycol, vegetable oils such as olive oil, and injectable organic esters such as ethyl oleate. Intravenous vehicles include fluid and nutrient replenishers, electrolyte replenishers (such as those based on Ringer's dextrose). The formulations may be presented in unit-dose or multi-dose kits, for example, ampules and vials, and may be stored in a freeze-dried (lyophilized) condition requiring addition of a sterile liquid carrier, for example, water for injections, prior to use.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, pastes, lotions, oils, or creams as generally known in the art.

For topical administration, for example, to skin, pharmaceutical compositions typically include ointments, creams, lotions, pastes, gels, sprays, aerosols, or oils. Carriers which may be used include Vaseline, lanolin, polyethylene glycols, alcohols, transdermal enhancers, and combinations thereof. An exemplary topical delivery system is a transdermal patch containing an active ingredient. For oral administration, pharmaceutical compositions include capsules, cachets, lozenges, tablets, or troches, as powder or granules. Oral administration formulations also include a solution or a suspension (e.g., aqueous liquid or a non-aqueous liquid; or as an oil-in-water liquid emulsion or a water-in-oil emulsion). For airway or nasal administration, pharmaceutical compositions can be formulated in a dry powder for delivery, such as a fine or a coarse powder having a particle size, for example, in the range of 20 to 500 microns which is administered in the manner by inhalation through the airways or nasal passage. Depending on delivery device efficiency, effective dry powder dosage levels typically fall in the range of about 10 to about 100 mg. Appropriate formulations, wherein the carrier is a liquid, for administration, as for example, a nasal spray or as nasal drops, include aqueous or oily solutions of the active ingredient.

For airway or nasal administration, aerosol and spray delivery systems and devices, also referred to as “aerosol generators” and “spray generators,” such as metered dose inhalers (MDI), nebulizers (ultrasonic, electronic, and other nebulizers), nasal sprayers and dry powder inhalers can be used. MDIs typically include an actuator, a metering valve, and a container that holds a suspension or solution, propellant, and surfactant (e.g., oleic acid, sorbitan trioleate, lecithin). Activation of the actuator causes a predetermined amount to be dispensed from the container in the form of an aerosol, which is inhaled by the subject. MDIs typically use liquid propellant and typically, MDIs create droplets that are 15 to 30 microns in diameter, optimized to deliver doses of 1 microgram to 10 mg of a therapeutic. Nebulizers are devices that turn medication into a fine mist inhalable by a subject through a face mask that covers the mouth and nose. Nebulizers provide small droplets and high mass output for delivery to upper and lower respiratory airways. Typically, nebulizers create droplets down to about 1 micron in diameter.

Dry-powder inhalers (DPI) can be used to deliver the compounds of the invention, either alone or in combination with a pharmaceutically acceptable carrier. DPIs deliver active ingredient to airways and lungs while the subject inhales through the device. DPIs typically do not contain propellants or other ingredients, only medication, but may optionally include other components. DPIs are typically breath-activated but may involve air or gas pressure to assist delivery.

Pharmaceutical formulations and delivery systems appropriate for the compositions and methods of the invention are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N.Y., pp. 253-315).

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.,” is derived from the Latin exempli gratia and is used herein to indicate a non-limiting example. Thus, the abbreviation “e.g.,” is synonymous with the term “for example.”

Definitions of common terms in cell biology and molecular biology can be found in The Encyclopedia of Molecular Biology, published by Blackwell Science Ltd., 1994 (ISBN 0-632-02182-9; Benjamin Lewin, Genes X, published by Jones & Bartlett Publishing, 2009 (ISBN-10:0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds. Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

FIGS. 1-6, and Extended FIGS. 1-9, as well as Examples 1-7 of U.S. Provisional Patent Application No. 63/434,125 is hereby specifically incorporated by reference.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Overview and Experimental Results

RTL8 is a poorly understood gag-like gene derived from the same Mart family of genes as the VLP-producing gene PEG1030. Based on the data presented here, Applicants proposed that RTL8 is an inhibitor of PEG10-derived VLP abundance. RTL8 inhibits VLP formation or release through incorporation into VLPs via RTL8: capsidNTD interactions, which is tolerated at low levels, but decreases VLP abundance in conditioned medium. Applicants proposed that high levels of RTL8 incorporation prevent the formation or release of VLPs from the cell, resulting in intracellular accumulation of PEG10.

It remains unknown precisely where RTL8 acts in the capsid assembly and release process to restrict PEG10 VLP abundance. One possibility is that upon RTL8 incorporation into early capsid multimers, the absence of an RTL8 capsidCTD prohibits the formation of higher-order assemblies generated through both homotypic CTD: CTD and heterotypic NTD: CTD interactions that are necessary for retroelement capsid assembly^{38-42,47}. A second possibility is that RTL8 incorporation disrupts proper localization of the PEG10 capsid or protein: protein interactions necessary for trafficking and release of VLPs. In support of this hypothesis, recent studies have shown that RTL8 influences the localization and function of UBQLN232, and RTL8 could similarly influence PEG10 localization. Further subcellular investigation of PEG10 capsid assembly and release is necessary to distinguish these possibilities.

Gag-like genes have been co-opted or exapted from retroelements multiple times in eukaryote evolution to restrict the infective capacity of retrotransposons or retroviruses^{17-21,48}. These genes derived from retroelement gag genes and act at the stage of capsid formation, release, or uncoating. The sheep gag-derived gene enJS56Al restricts infection by the enJSRV family of viruses by disrupting capsid assembly and release^{17,18}. The mouse Fvl gene is a gag-like restriction factor that targets incoming murine leukemiavirus (MuLV) particles by coating the endocytosed virus and preventing disassembly of the capsid^{21,48}. In a mechanism that closely mimics the PEG10: RTL8 relationship, the Ty1 retrotransposon in yeast is tightly regulated by the presence of a cryptic start site within the gag open reading frame, which results in the production of a truncated capsidCTD fragment. The truncated fragment prevents homotypic CTD: CTD interactions of intact capsid, thereby limiting Tyl re-integration^{19,20}. In each system, the gag-like genes use an affinity for capsid to inhibit the formation, release, or uncoating of the infectious particle. There are many Gag-like genes in the human genome^{8,12}, but no such gag-like restriction mechanisms have been described in humans. The data outlined here describe a similar phenomenon between the human genes RTL8 and PEG10, with the important distinction that PEG10 is not capable of replication and is a domesticated gene with a documented role in placental reproduction^{4,25}. Based on our data, Applicants posited that RTL8 can be included in this list of these ‘gag decoys’ which act at the step of capsid formation or release.

The ability of RTL8 to antagonize PEG10-derived VLPs may be particularly relevant when considering the utility of PEG10 in generating biocompatible mRNA delivery systems, such as SEND¹⁵. Generation of PEG10-derived VLPs for the purposes of nucleic acid delivery in human cells may be limited to cell lines expressing abundant RTL8. Further, RTL8 may contaminate PEG10-derived VLPs due to low levels of incorporation. Removal of RTL8 expression was sufficient to increase VLP yield in HepG2 cells; however, the same manipulation had no effect hTR-8 cells. Therefore, careful selection of cell line is essential to maximize VLP release. For the purposes of maximizing PEG10-derived VLP yield, genetic modification of cell lines with the suppression or deletion of RTL8 may maximize their utility as production lines. As such, in some embodiments of the current invention, inhibition or deletion of UBQLN2 may also increase in PEG10 VLP formation.

### Example 2: PEG10 is Spontaneously Released as Virus-Like Particles in Some Human Cell Lines

Endogenous and transfected human PEG10 have been observed to form VLPs that are released into cell culture medium^{5,15,16}, but the universality of PEG10-derived VLP production in human cells was unknown. To generate a more complete understanding of PEG10 VLP production in a native context, Applicants quantified VLP abundance in conditioned medium from a range of cultured human cell lines. Applicants tested the abundance of PEG10 in cell lysate (FIG. 1a-b) and conditioned medium (FIG. 1c-d) following enrichment of extracellular particles by ultracentrifugation. PEG10 abundance in lysate and the VLP fraction was measured by western blot using a polyclonal antibody which detects the short (‘gag’) and long (‘gag-pol’) forms of PEG10. This permits the separate quantitation of gag and gag-pol proteins, as well as their respective contribution to VLPs, in each cell line (FIG. S1a-c).

All cell lines showed detectable levels of PEG10 gag and gag-pol protein from cell lysate (FIG. 1a-b). In contrast, gag and gag-pol proteins were only detectable in the VLP fraction of some cell lines (FIG. 1c-d): notably, HepG2 and hTR-8 cells. HepG2 cells had the highest lysate abundance and VLP production of all cell lines tested. However, across all cell lines, lysate levels of PEG10 did not strongly correlate with VLP abundance (FIG. S1c). HEK293 cells had intermediate lysate abundance (FIG. 1a) but very little in the VLP fraction (FIG. 1c). In contrast, hTR-8 cells had intermediate lysate abundance (FIG. 1a) and high levels of VLPs (FIG. 1c). HepG2 and hTR-8 cell lines exhibited the highest ratio of gag-pol: gag protein amongst all cell lines tested (FIG. S1d), indicating a possible role for the gag-pol form of PEG10 in regulation of VLP yield.

To further purify PEG10-derived VLPs, a standard iodixanol fractionation was performed. First, fractionation was performed on conditioned medium from HepG2 cells. ALIX, a marker of exosomes²⁹, was detected along with Tubulin in the fractions corresponding to 15% iodixanol (FIG. 1e). PEG10 gag-pol and gag were first detected in the fractions corresponding to 25% iodixanol, before ALIX or tubulin were evident (FIG. 1e); however, PEG10 was also abundant in ALIX-positive fractions (FIG. 1e). Iodixanol fractionation of conditioned medium from transfected HEK293 cells, which do not normally release PEG10-derived VLPs, also displayed the presence of HA-tagged PEG10 in the 25% and 15% fractions (FIG. S2b), indicating that transfected, HA-tagged PEG10 can form VLPs that sediment similarly to endogenously formed PEG10-derived VLPs.

### Example 3: RTL8 Incorporates into PEG10 VLPs and Decreases the Efficiency of Release

PEG10 is one of a family of closely related gag-like genes in humans that are collectively referred to as the Mart family^{30,31}. Previous studies have implicated a relationship between the Mart genes PEG10 and RTL^{85,28,32}, previously known as Cxx1 or FAM127. RTL8A, RTL8B, and RTL8C are three nearly identical members of the Mart family that encode a truncated gag-like protein with high levels of homology to PEG10³⁰. In particular, amino acid alignment of RTL8 with PEG10 shows that RTL8 bears a strong resemblance to the CA^{NTD} portion of PEG10 gag (FIG. 2a, FIG. S3a).

Due to the structural similarity³⁰ and reported interactions between RTL8 and PEG10^{33,34} Applicants hypothesized that RTL8 may incorporate into PEG10 VLPs. First, iodixanol-fractionated HepG2 VLPs were probed with an anti-RTL8 antibody, but no signal was observed at the expected molecular weight (not shown). Next, Applicants tried an overexpression approach. HEK293 cells were used as a model because of their ease of transfection and their lack of natural PEG10 VLP production, which would interfere with interpretation of VLP formation from transfected constructs. FLAG-RTL8c was co-expressed in HEK293 cells with empty vector or with HA-PEG10 gag-pol, followed by analysis of the VLP fraction in ultracentrifuged media by western blot. FLAG-RTL8c was only found in the VLP fraction when HA-PEG10 was co-expressed (FIG. 2b), indicating that RTL8 is unable to independently form VLPs but can incorporate into VLPs produced by PEG10. eGFP recovery from ultracentrifuged media was unchanged with PEG10 co-expression (FIG. S3b) indicating that RTL8c's presence in the VLP fraction is not due to nonspecific PEG10-dependent packaging. The presence of low levels of eGFP in ultracentrifuged medium was consistent with iodixanol fractionation: eGFP recovered from conditioned medium was present in ALIX-positive fractions but at minimal intensity in the 25% iodixanol fractions (FIG. S3c).

To determine an approximate ratio of PEG10: RTL8 abundance in ultracentrifuged VLPs, HA-tagged PEG10 was expressed along with an HA-tagged RTL8c. The relative abundance of HA-RTL8c was decreased in the VLP fraction compared to cell lysate: upon co-expression, RTL8 is found at approximately 15% the abundance of PEG10 in the lysate but is approximately 5% the abundance of HA-PEG10 in the VLP fraction (FIG. S3d-e). Together, they indicate that either RTL8 incorporation into PEG10-derived VLPs is inefficient, or that only low levels of RTL8 incorporation are tolerated before VLP formation or release is impossible. Co-transfection of HA-tagged PEG10 with FLAG-tagged RTL8c caused no gross changes to gag-pol or gag abundance in cell lysate (FIG. 2c, left). In contrast, co-expression of RTL8c decreased the overall abundance of PEG10 VLPs in conditioned media (FIG. 2c, right). This apparent difference was not significant when both gag and gag-pol bands were summed to approximate VLP abundance (FIG. 2d, left), but when just the gag form of PEG10 was transfected, the addition of RTL8c significantly decreased VLP abundance in the medium (FIG. 2d, right).

The decrease in VLP abundance of PEG10 upon co-transfection suggested that RTL8 may inhibit VLP formation or release. Further quantification of PEG10 abundance in cell lysate showed a significant increase in intracellular PEG10 when PEG10 gag-pol was co-expressed with RTL8c (FIG. S4b). To examine the retention of intracellular PEG10 using an orthogonal approach, a flow cytometry-based fluorescent reporter of PEG10 abundance was used. Flow cytometry allows for high-throughput quantitation of intracellular protein content with two notable improvements on traditional western blot techniques: 1) analysis of only the transfected cell population, thanks to a fluorescent reporter of transfection efficiency, and 2) normalization of intracellular protein content to transfection efficiency, using the same reporter, on a per-cell basis. PEG10 was fused at the C-terminus to the fluorophore Dendra235 as a reporter for PEG10 abundance, with an IRES-eCFP cassette as a transfection efficiency control (FIG. 2e). Fusion of PEG10 with Dendra2 did not interfere with VLP production, as a VLP prep of PEG10-Dendra2 was positive for PEG10 (FIG. S4c). Co-expression of the PEG10 reporter construct with FLAG-RTL8c led to increased intracellular fluorescence as measured by flow cytometry (FIG. 2f), consistent with intracellular accumulation of PEG10 due to RTL8 restriction and supporting the hypothesis that RTL8 inhibits either PEG10 VLP assembly or release.

Given that RTL8 closely resembles the N-terminus of PEG10, Applicants speculated that RTL8 restricts VLP abundance by mimicking PEG10's N-terminus and competitively incorporating into a growing capsid. In this model of interference, co-transfection of a truncated PEG10 capsid^{NTD} with full-length PEG10 should similarly restrict VLP abundance. PEG10-Dendra2 was co-expressed with an unlabeled capsid^{NTD}, resulting in similar levels of intracellular PEG10-Dendra2 retention as compared to RTL8 co-expression (FIG. 2f). These data support a model where RTL8 acts as a competitive inhibitor of PEG10 capsid assembly.

### Example 4: RTL8 Interacts with the N-Terminal Lobe of the PEG10 Capsid Domain

To determine whether RTL8 was inhibiting VLP release through protein-protein interactions, Applicants expressed HA-PEG10 and FLAG-RTL8c in HEK293 cells and performed crosslinking co-immunoprecipitation. Both proteins were readily visualized by western blot of cell lysate (FIG. 3a). HA-PEG10 was visible as four distinct bands representing gag-pol, gag, and two self-cleavage products generated via activity of the PEG10 protease domain^{16,36,37}. The 37 kDa band represents a retrovirus-like capsid fragment, and the 22 kDa band reflects a protein fragment consisting of the N-terminal ~100 amino acids of the protein (referred to as the ‘N-terminal fragment’, or ‘NTF’) 16. Upon immunoprecipitation of HA-PEG10, Applicants observed co-immunoprecipitation of FLAG-RTL8c (FIG. 3a). When Applicants performed the reciprocal experiment (IP-FLAG), Applicants observed co-immunoprecipitation of HA-PEG10 gag-pol, gag, and capsid, but not the shorter proteolytically cleaved N-terminal fragment (FIG. 3b), indicating that while gag and capsid are sufficient to interact with RTL8c, the very N-terminal region of PEG10 is insufficient.

Retroelement capsid proteins have two distinct lobes: the N-terminal lobe (capsid^{NTD}) and C-terminal lobe (capsidCTD)³⁸. During retroelement capsid assembly, NTD lobes self-associate into penta- and hexameric cones, which are held together by homotypic CTD: CTD interactions on adjacent cones³⁸⁻⁴². Based on sequence alignment and structural modeling, PEG10 capsid closely resembles that of the ancestral Ty3 retrotransposon (FIG. S5a-b). Indeed, recent structural studies confirm that PEG10 capsid^{CTD} closely resembles ancestral Ty3 capsid and is capable of dimerization⁴³.

To more precisely map the interaction between PEG10 and RTL8, Applicants generated PEG10 capsid truncation constructs representing the N- and C-terminal lobes (HA-capsid^{NTD} and HA-capsid^{CTD}, FIG. 3c). Constructs were co-expressed with FLAG-RTL8c, and FLAG was immunoprecipitated. FLAG-RTL8c interacts with HA-PEG10 gag-pol, gag, and capsid^{NTD}, but not capsid^{CTD} (FIG. 3d, FIG. S5c), indicating that the NTD lobe is both necessary and sufficient for interaction with RTL8. Co-immunoprecipitation of HA-gag and HA-capsid^{NTD} with FLAG-RTL8c results in the appearance of a higher molecular weight band that stains positive for both FLAG and HA (FIG. 3d, FIG. S5c, asterisks). This band is at the correct molecular weight to be the crosslinked dimer of PEG10 with RTL8, which supports the hypothesis that these proteins interact in the cell.

It was surprising that FLAG-RTL8c was unable to co-immunoprecipitate the small N-terminal fragment (NTF) of PEG10 (FIG. 3b) given that the slightly larger HA-capsid^{NTD} construct was sufficient (FIG. 3d). Truncation constructs of capsid were generated that mimic the natural self-cleavage of PEG10 and were co-expressed with FLAG-RTL8c (FIG. S6a), followed by co-IP. Neither HA-NTF nor HA-CTF were capable of interaction with FLAG-RTL8c (FIG. S6b), consistent with results in FIG. 3e and suggesting that the entirety of the capsid^{NTD} lobe is necessary for interaction between PEG10 and RTL8.

Alphafold multimer modeling of a putative PEG10: RTL8 interaction further supports the role of the PEG10 NTD in facilitating an interaction of the two proteins. Consistent with an NTD: NTD-like model of binding, an Alphafold prediction showed close interaction between RTL8 and the NTD of PEG10 (FIG. 3e). Residues predicted to interact were predominantly in the second α-helix of RTL8, located between residues 41-55, and the third and fourth «-helices of PEG10 gag, located between residues 110-135 (FIG. 3e). In contrast, PEG10 self-association appears to favor a CTD: CTD-like interaction, with NTDs left unbound (FIG. S6c). If PEG10 CTDs are removed to force an NTD: NTD-like interaction, the modeled dimer closely resembles the RTL8: PEG10 interface, with the third and fourth-helices of both NTD domains driving dimerization (FIG. S6d). An overlay of this NTD: NTD model on top of the RTL8: PEG10 model shows agreement of the two structures, with the third-helix of PEG10 resting within the second α-helix of RTL8 (FIG. S6e). Based on these data, as well as the structural homology of PEG10 capsid^{NTD} and RTL8, Applicants conclude that the PEG10: RTL8 interaction likely resembles the homotypic capsid NTD: NTD interactions typical of the retroelement capsid assembly process.

### Example 5: The Intracellular Retention of PEG10 by RTL8 Shows Species, but not Paralog Specificity

Three paralogs of RTL8 (RTL8a, b, and c) are all found on the X-chromosome and share >92% identity³⁰. Applicants compared all three RTL8 paralogs for the ability to induce retention of intracellular PEG10-Dendra2 and saw no differences between RTL8 genes (FIG. 4a), indicating that each form of RTL8 is capable of promoting the retention of PEG10.

One hallmark of gag-based restriction of retroelement capsids is the specificity of interaction. In mice, different alleles of the gag-like gene Fvl show differential ability to inhibit unique strains of MuLV^{44,45}. Like humans, mice express three Rtl8 paralogs which share considerable homology to human RTL8 genes³⁰. Mice also express Peg10, which diverges from human in the expansion of a proline and glutamine-rich region near the C-terminus³⁰. Mouse FLAG-Rtl8b, which shares the highest homology to human RTL8 (FIG. 4b), bound less efficiently to human PEG10 by co-immunoprecipitation (FIG. 4c). Further, co-expression of mouse Rtl8b had no effect on the intracellular retention of human PEG10-Dendra2 by flow cytometry (FIG. 4d). Conversely, mouse PEG10 was capable of binding both human and mouse FLAG-RTL8 (FIG. 4e) but its intracellular levels were unaffected by either (FIG. 4f). In conclusion, the RTL8 effects on PEG10 intracellular and VLP abundance are only evident using human genes.

### Example 6: RTL8 Inversely Correlates with PEG10 VLP Abundance in Two Cohorts of Cell Lines

Given the ability of RTL8 to inhibit PEG10-derived VLP release upon transfection, Applicants speculated that endogenous expression of RTL8 may influence the ability of cells to spontaneously release VLPs. Endogenous protein levels of RTL8 in the cell lines tested in FIG. 1 were very low and could only be visualized and quantified using sophisticated western blot imaging equipment (FIG. 2). Though the cohort of cell lines is small, careful quantitation of RTL8 from cell lysate showed that high intracellular RTL8 abundance tended to correlate inversely with PEG10-derived VLP abundance in conditioned medium (FIG. 2).

### Example 7: Natural Production of PEG10 VLPs can be Enhanced Through Modulation of RTL8

The utility of PEG10-derived VLPs for biotechnological use relies on the ability to produce VLPs in high quantities. The data presented here support a model whereby RTL8 inhibits PEG10-derived VLP release from cells (FIG. 2); therefore, modulation of RTL8 may improve VLP yield for the purposes of large-scale production. Applicants sought to increase natural levels of PEG10 VLP production by silencing RTL8 expression in HepG2 cells, which spontaneously release PEG10 VLPs (FIG. 1). Because of the similarity between RTL8a, RTL8b, and RTL8c, Applicants sought to silence all RTL8 genes simultaneously with an siRNA approach. RTL8 siRNA robustly diminished RTL8 protein levels in cell lysate (FIG. 6a-b) without changing lysate levels of PEG10 (FIG. 6a-d). However, siRTL8-treated cells showed approximately twice as much PEG10 in the VLP fraction (FIG. 6a,e), suggesting that the effect of RTL8 is to specifically regulate VLPs. This effect was cell-type specific, as modulation of hTR-8 cells with the same siRNA construct showed no change to VLP abundance (FIG. S7). In conclusion, the abundance of PEG10-derived VLPs is remarkably cell-type specific and likely involves many factors, including PEG10 expression level, RTL8 expression, and other regulatory components that remain poorly understood.

### Example 8: UBQLN2 Regulates PEG10 Gag-Pol Abundance and VLP Release in a Human Trophoblast Cell Line

Elevation of the ratio of gag-pol: gag in HepG2 and hTR-8 cells suggested that a component of the pol open reading frame may increase the efficiency of PEG10-derived VLP formation and/or release. Recent studies have shown the proteasome shuttle factor ubiquilin 2 (UBQLN2) specifically regulates the abundance of PEG10 gag-pol by targeting it for proteasomal degradation. To test whether UBQLN2 similarly regulates VLP production, the present inventors stably introduced shRNAs targeting UBQLN2 to HepG2 and hTR-8 cells (FIG. S8a,b, Extended Data FIG. 9). Consistent with previous work, knockdown of UBQLN2 in hTR-8 cells led to an approximately two-fold increase in PEG10 gag-pol abundance in cell lysate, independent of changes to gag (FIG. S8a,c-d). In hTR-8 cells, knockdown of UBQLN2 also resulted in a two-fold increase in VLP abundance (FIG. S8e,f). The loss of UBQLN2 in HepG2 cells did not result in changes to lysate gag, gag-pol, or PEG10 VLP levels (Extended Data FIG. S8b-e), which may be due to a large excess of PEG10 over UBQLN2, or additional regulatory factors masking a UBQLN2-dependent effect.

### Example 9: RTL8 incorporates into PEG10 VLPs and decreases the efficiency of release

Due to the structural similarity (FIG. 5a) and demonstrated interaction between RTL8 and PEG10 capsid (FIG. 12), the present inventors hypothesized that RTL8 can incorporate into PEG10 VLPs. RTL8c was expressed in HEK293 cells alone and with PEG10, followed by analysis of the VLP fraction in ultracentrifuged media by western blot. RTL8 was only found in the VLP fraction when PEG10 was co-expressed (FIG. 5b), indicating that RTL8 is unable to independently form VLPs but can incorporate into VLPs produced by PEG10. RTL8 exists at approximately 5% the abundance of PEG10 in the VLP fraction (Extended Data FIG. S13b,c), indicating either that RTL8 incorporation into PEG10-derived VLPs is inefficient, or that only low levels of RTL8 incorporation are tolerated before VLP formation or release is impossible. Consistent with the latter possibility, co-expression of RTL8c also decreased the overall abundance of PEG10 VLPs in conditioned media (FIG. 5c,d). The inhibitory effect was more pronounced against forms of PEG10 lacking a functional pol region: gag alone, or a protease dead mutant (gag-pol^{ASG}), had a larger magnitude of change when co-expressed with RTL8 despite higher baseline abundance of VLPs (Extended Data FIG. 4f-h).

If RTL8 inhibits VLP assembly or release from cells, co-expression of PEG10 with RTL8 should lead to accumulation of intracellular PEG10 due to the decreased export of PEG10 in VLPs. The present inventors tested retention of intracellular PEG10 with a flow cytometry-based fluorescent reporter of PEG10 abundance (Extended Data FIG. 5d). As proof of principle, the present inventors overexpressed PEG10 in HEK293^{WT} and HEK293^{TKO} cells and saw an increase in fluorescence in cells lacking UBQLN1, 2, and 4 (Extended Data FIG. 5e). Co-expression of the PEG10 reporter construct with RTL8c also led to increased intracellular fluorescence (FIG. 5e,f), consistent with intracellular cytosolic accumulation of PEG10 due to RTL8 restriction and furthering the hypothesis that RTL8 inhibits either PEG10 VLP assembly or release.

### Example 10: Materials and Methods

### Cloning

All cloning was performed by Gibson assembly (Gibson HiFi master mix, Invitrogen) and transformed into chemically competent DH5a E. coli cells (Invitrogen). Transformed E. coli were plated on either 50 mg/mL kanamycin (Teknova) or 100 mg/mL carbenicillin (Gold Biotechnology) LB agar (Teknova) plates overnight at 37° C. Single colonies were picked and grown overnight in 5 mL LB Broth (Alfa Aesar) with kanamycin or carbenicillin at 37° C. with shaking at 220 rpm. Shaking cultures were mini-prepped (Zymo) and sent for Sanger Sequencing (Azenta). Sequence verified samples were then grown in 50 mL LB Broth overnight with appropriate antibiotic at 37° C. with shaking at 220 rpm. 50 mL cultures were midi-prepped (Zymo) for transfection.

### Cell Lines

hTR-8/SVneo (CRL-3271), SK-N-SH (HTB-11), T98G (CRL-1690), CCF-STTG1 (CRL-1718), BE (2)-M17 (CRL-2267), and M059K (CRL-2365) cells were purchased from ATCC. A549 and U-87 MG cells were a gift from Dr. Roy Parker (Department of Biochemistry, CU Boulder). HepG2 cells were obtained from ATCC (HB-8065) via the CU Boulder Biochemistry Shared Cell Culture Facility. HEK293 cells were a gift from Dr. Ramanujan Hegde (Medical Research Council Laboratory of Molecular Biology, Cambridge England).

All cells were maintained at 37° C. with 5% CO₂. HEK293 and A549 cells were maintained in DMEM (Invitrogen) supplemented with 100 U/mL Penicillin-Streptomycin (Invitrogen), 1% L-glutamine (R&D Systems, Inc.), and 10% FBS (Millipore Sigma). BE (2)-M17 cells were cultured in DMEM/F12 (Invitrogen) containing 10% FBS and Penicillin-Streptomycin. M059K cells were cultured in DMEM/F12 (Invitrogen) containing 0.05 mM NEAA (Invitrogen), 20% FBS and Penicillin-Streptomycin. hTR-8/SVneo cells were maintained in RPMI 1640 (Invitrogen) supplemented with Penicillin-Streptomycin, L-glutamine, and 10% FBS. CCF-STTG1 cells were cultured in RPMI-1640 (Cytiva Life Sciences) containing 20% FBS and Penicillin-Streptomycin. HepG2 and U87 MG cells were maintained in MEM (Invitrogen) supplemented with Penicillin-Streptomycin, L-glutamine, and 10% FBS. SK-N-SH and T98G cells were cultured in MEM/EBSS (Cytiva Life Sciences) containing 1 mM sodium pyruvate (Invitrogen), 0.1 mM NEAA, 10% FBS and Penicillin-Streptomycin.

### Transfection

Cells were grown to 70% confluency and transfected with Lipofectamine 2000 (ThermoFisher) according to manufacturer's instructions. For 6-well plates, 2.5 μg plasmid DNA was transfected per well. For 12-well plates, lug plasmid DNA was transfected per well. For 96-well plates, 0.1 μg plasmid DNA was transfected per well. For cotransfections, equal mass amounts of each plasmid were added to the total amount listed above. Transfection mixture was prepared at a ratio of lug DNA: 2.5 μL Lipofectamine 2000. Unless otherwise stated, cells and media were harvested 48 h following transfection.

### Generation of Stable Cell Lines

MISSION lentiviral packaging plasmids and shRNA plasmids targeting UBQLN2 were purchased from Sigma-Aldrich. Generation of Lentiviral delivery vectors was performed according to manufacturer's recommendation. Briefly, ~1.7×10⁶ 0Invitrogen). The following day, virus-containing media was collected, filtered through a 0.45 μm filter, and stored at −80° C. until infection. Lentiviral infection was performed via spinfection of cells on two consecutive days with virus-containing medium. Two days after infection, selection for infected cells was initiated through the introduction of 2 mg/mL (hTR-8/SVneo) or 8 mg/mL (HepG2) puromycin. Once stably infected, cells were maintained in growth media supplemented with puromycin.

### Virus-Like Particle Isolation

Crude preparation: For endogenous VLP production, T75 flasks were plated at 70% confluency. For overexpression experiments, cells were plated at 70% confluency in 6-well plates and were transfected as described above. Cells were grown for 48 hr and conditioned media was collected. Media was first centrifuged at 2700×g for 10 min to remove cellular debris. The supernatant was harvested, then spun by ultracentrifugation using a preparative ultracentrifuge (Beckman Coulter) at 134,000×g (Beckman SW41Ti rotor) for 4 hr at 4° C. over a 30% sucrose (MP Biomedicals) cushion. Media and sucrose were aspirated, and the VLP-containing pellet was resuspended in lysis buffer for western blot.

Iodixanol preparation: Iodixanol (Optiprep, Sigma Aldrich) gradients were prepared using PBS-MK buffer to generate fractions of 60%, 40%, and 25% iodixanol in PBS-MK, and 15% iodixanol in PBS-MK with 1M NaCl. The 60% and 25% fractions were pre-mixed with phenol red to color the gradient. Iodixanol steps were layered in Beckman Coulter 38.5 mL open-top, thin-wall ultra-clear tubes using a needle and 10 mL syringe. 5 mL of the 60% step, 5 mL of the 40% step, 6 mL of the 25% step, and 8 mL of the 15% step were sequentially layered. Conditioned medium was spun at 2,700g for 15 minutes at 4° C. to remove cell debris and up to 8 mL of the supernatant was layered on top of iodixanol. Then, PBS was added to balance the tubes in an SW32 Ti rotor. Media was spun at 120,000g for 18 hours at 4° C.

After spinning, parafilm was put over the top of the tube to seal and an 18 gauge needle (BD) was used to puncture just below the 60%-40% interface. Drops were collected into microcentrifuge tubes in approximately 1 mL fractions, starting with #1. Color change and change in flow rate was used to denote the approximate locations of iodixanol steps across collected fractions.

Protein was isolated from fractions using methanol:chloroform precipitation and 500 μL of proteinaceous starting material. After precipitation, protein was resolubilized in 12 μL of 8M urea buffer to which 3 μL of 5× Laemmli sample buffer was added. The entire sample was run on a 4-12% Bis-tris gel and western blotted according to methods.

### Human Brain Tissue

Postmortem human brain tissue was obtained from the Michigan Brain Bank. 40 μg of brain tissue homogenate as determined by a BCA assay was used for western blotting.

### Western Blotting

Cells were harvested by trypsinization, pelleted, and washed in PBS. For bulk cell lysate analysis, cell pellets were resuspended in in 8M urea (Fisher Chemical) containing 75 mM NaCl (Honeywell), 50 mM HEPES (Millipore Sigma) pH 8.5, with 1× complete Mini EDTA-free protease inhibitor (Roche) and incubated at room temperature for 15 min. Lysate was cleared by centrifugation at 21,300×g and the supernatant collected for western blot. Total protein was quantified by BCA assay (Pierce).

For endogenous protein analysis, 15 μg total protein was loaded into NuPage 4-12% Bis-Tris precast protein gels (Life Technologies) per sample. For overexpression experiments, 4 μg total protein was loaded per sample. Samples with equal protein content were generated by adding excess urea buffer to that all samples were of equivalent volume. Then, 5× Laemmli sample buffer supplemented withβME (Sigma Aldrich) was added to a final concentration of 1×. Proteins were separated by SDS-PAGE using 1× NuPage MES SDS running buffer (Life Technologies). For samples lysed in urea, wells of the protein gel were equilibrated in urea lysis buffer for 10 min, and urea washed out before loading protein samples.

Proteins were transferred to nitrocellulose membrane (Amersham) using the Invitrogen Mini Blot Module according to manufacturer's instructions. Membranes were blocked with Intercept (PBS) blocking buffer (LICOR) for 30 min at room temperature. Membranes were incubated with primary antibody overnight at 4° C. Membranes were incubated with secondary antibodies for 30 min at room temperature. After both antibody incubations, membranes were washed three times for 5 min each in TBST. Membranes were imaged on a LICOR Odyssey CLx and analyzed with LICOR ImageStudio software.

Unless otherwise stated, western blots were analyzed as follows. For cell lysate, target protein signal was normalized to tubulin as a loading control. This ratio was then normalized to the mean of the ratio for all samples within the experiment to account for technical variability across independent experiments. For endogenous VLP production, target protein signal was normalized to the mean target signal for all samples within the experiment. For VLP production from transfected PEG10, the overexpressed target protein signal from the VLP fraction was normalized to the tubulin-normalized cell lysate abundance of that target to account for variability in transfection efficiency. This ratio was then normalized to the mean of the ratio for all samples within the experiment to account for technical variation across independent experiments.

### Crosslinking Co-Immunoprecipitation

Cells were harvested by pipetting in ice cold PBS and centrifuged at 300×g for 3 min, then resuspended in 300 μL 0.1% PFA in PBS and incubated for 7 min at room temperature to crosslink proteins. Cells were collected by centrifugation at 300×g for 3 min and washed three times with ice cold PBS. Crosslinked cells were lysed in 150 μL lysis buffer containing 1% Triton X-100 (Sigma), 100 mM NaCl (Honeywell), 10 mM Hepes pH 7.5 (Sigma), 10 mM EGTA (Sigma), 10 mM EDTA (Sigma), and 1× protease inhibitor (Roche), and incubated for 30 min on ice. Lysate was pre-cleared by centrifugation at 16,000×g for 5 min at 4° C. and the supernatant was collected for immunoprecipitation.

Beads were first crosslinked to antibodies. Primary antibodies (5 μg) or isotype controls (5 μL) were incubated with 40 μL protein A (Invitrogen) or protein G (Pierce) bead slurry for 30 min to couple antibodies to beads. After coupling, beads were collected by centrifugation and the supernatant discarded. Unless otherwise stated, all incubations and washes were performed with end-over-end rotation at room temperature. All centrifugation to collect beads was performed 2,500×g for 2 min. Beads were washed twice (1 mL 0.2M sodium borate, pH 9 for 1 min), resuspended in 400 μL 20 mM dimethyl pimelimidate (DMP, Thermo) and incubated for 30 min to crosslink antibodies. After crosslinking, beads were washed twice (1 mL 0.2M ethanolamine (EMD Millipore) pH 8 for 5 min) and incubated in 1.4 mL 0.2M ethanolamine for two hr. Beads were then washed twice (1 mL PBS for 5 min).

50 μL crosslinked cell lysate and 50 μL Triton lysis buffer were added to antibody-conjugated beads and incubated overnight at 4° C. with end-over-end rotation. After incubation, beads were collected by centrifugation and washed three times (500 μL PBS, 0.1% Triton X-100 for 2 min). To elute proteins, beads were resuspended in elution buffer (20 μL 5× Laemmli samle buffer and 30 μL PBS) and incubated at 90° C. for 10 min. Following elution, the samples were centrifuged to collect the beads and the supernatant removed for analysis by western blot as described above. For SDS-PAGE, 7.5 μL protein lysate (5%) and 25 μL (50%) immunoprecipitated sample were loaded.

### Sequence Alignment

Sequence alignment was performed on the EMBL-EBI MUSCLE online interface50. Alignments used the ClustalOmega algorithm51 using the default parameters.

### Structure Prediction

Structures of Homo sapiens PEG10 gag (AA 1-325) and RTL8 (AA 1-113) were modeled using the Phyre 2.0 web server⁵² using the intensive modeling mode. Structures were visualized using UCSF Chimera⁵³. Dimers of RTL8: PEG10 and PEG10: PEG10 were modeled using Alphafold2 with MMseqs2 at ColabFold⁵⁴ v1.5.2 found at: colab.research.google.com/github/sokrypton/ColabFold/blob/main/AlphaFold2.ipynb #scrollTo=kOblAo-xetgx.

The entire protein sequence of RTL8c with the entire gag sequence (AA1-325) of PEG10 were used to model dimer structures of Homo sapiens RTL8 with Homo sapiens PEG10. Two sequences of PEG10 gag (AA1-325) were used to model PEG10 gag dimerization, and two sequences of PEG10 CA^{NTD} (AA1-154) were used to model NTD: NTD interactions. No template was used, and all standard settings were selected. The resulting.pdb structures were visualized in Pymol. AA1-73 of PEG10, including the first alpha helix of the protein, were rendered invisible for all figures. For RTL8: PEG10 visualizations, the CACTD was rendered invisible.

### Flow Cytometry

HEK293 cells were transfected in a 96-well or 48-well plate with plasmids encoding a PEG10-Dendra2 fusion protein followed by an IRES-CFP cassette under control of a CMV promoter. Cells were lifted by pipetting in FACS buffer (D-PBS, 2% FBS, 0.1% sodium azide) and analyzed on a FACSCelesta (BD Biosciences). At least 20,000 events were collected per sample on the cytometer. Flow cytometry analysis was performed using Flowjo software (Treestar). Single cells were first gated on FSC-A vs SSC-A, followed by gating of CFP+ cells. To account for transfection efficiency, an algebraic parameter was generated of the mean fluorescence intensity (MFI) of PEG10-Dendra2 divided by the MFI of IRES-CFP for each event. To generate summary data, the geometric mean of the custom algebraic parameter was generated for each sample.

### Human Protein Atlas

Data on RTL8 and PEG10 mRNA expression in human tissues was taken from the RNA Expression Consensus dataset containing data from The Human Protein Atlas and GTEx transcriptomics. RNA consensus tissue gene data was downloaded from the Human Protein Atlas and is based on The Human Protein Atlas version 23.0 and Ensembl version 109.

### Statistics

Error bars represent mean±SEM for all FIG.s. All statistical analysis was performed using GraphPad Prism software. Standard one-way ANOVA was corrected for multiple comparisons by Dunnett's test as recommended. Standard two-way ANOVA was corrected for multiple comparisons by Šídák's test as recommended. For all figures, statistical tests are listed in the figure legend and *p<0.05, ** p<0.01, *** p<0.001, and **** p<0.0001.

TABLE 1
Antibodies

| Target | Clonality | Species | Vendor | Catalog # | Dilution | Application |
|---|---|---|---|---|---|---|
| PEG10 | Polyclonal | Rabbit | Proteintech | 14412-1-AP | 1:1000 | WB |
| FAM127B (RTL8) | Polyclonal | Rabbit | Proteintech | 20282-1-AP | 1:1000 | WB |
| FLAG | Monoclonal | Mouse | Sigma | F3165 | 1:5000 | WB, IP |
| | M2 | | | | | |
| FLAG | Monoclonal | Rabbit | CST | 14793 | 1:5000 | WB |
| | D6W5B | | | | | |
| HA | Monoclonal | Mouse | Sigma | H3663 | 1:5000 | WB, IP |
| | HA-7 | | | | | |
| HA | Monoclonal | Rabbit | CST | 3724 | 1:5000 | WB |
| | C29F4 | | | | | |
| GAPDH | Monoclonal | Rabbit | CST | 2118 | 1:5000 | WB |
| | 14C10 | | | | | |
| Tubulin | Monoclonal | Mouse | Novus | NB100-690 | 1:10000 | WB |
| | DM1A | | | | | |
| ALIX | Monoclonal | Mouse | CST | 2171S | 1:1000 | WB |
| | 3A9 | | | | | |
| a-mouse IgG 680 | Polyclonal | Goat | Licor | 926-68070 | 1:20000 | WB |
| a-mouse IgG 800 | Polyclonal | Goat | Licor | 926-32210 | 1:20000 | WB |
| a-rabbit IgG 680 | Polyclonal | Goat | Licor | 926-68071 | 1:20000 | WB |
| a-rabbit IgG 800 | Polyclonal | Goat | Licor | 926-32211 | 1:20000 | WB |

TABLE 2
Constructs

| | | | | NCBI protein | |
|---|---|---|---|---|---|
| Construct Name | Insert | Tag | Species | reference | Vector |
| eGFP | eGFP | 2x HA (N-terminal) | Aequorea | 6XZF_A | pcDNA3.1 |
| | | | victoria | | |
| gag-pol | PEG10 (AA1-708) | 2x HA (N-terminal) | Homo sapiens | NP_055883.2 | pcDNA3.1 |
| gag | PEG10 (AA1-325) | 2x HA (N-terminal) | Homo sapiens | NP_001035242.1 | pcDNA3.1 |
| capsid^{NTD} | PEG10 (AA1-160) | 2x HA (N-terminal) | Homo sapiens | | pcDNA3.1 |
| capsid^{CTD} | PEG10 (AA161-259) | 1x HA (C-terminal) | Homo sapiens | | pcDNA3.1 |
| N-terminal | PEG10 (AA1-111) | 2x HA (N-terminal) | Homo sapiens | | pcDNA3.1 |
| fragment | | | | | |
| C-terminal | PEG10 (AA112-325) | 2x HA (C-terminal) | Homo sapiens | | pcDNA3.1 |
| fragment | | | | | |
| FLAG- | RTL8a (AA1-113), with | 3x FLAG (N- | Homo sapiens | NP_001071640.1 | pcDNA3.1 |
| HsRTL8a- | 700bp 3′UTR | terminal) | | | |
| 3′UTR | | | | | |
| FLAG- | RTL8b (AA1-113), with | 3x FLAG (N- | Homo sapiens | NP_001071641.1 | pcDNA3.1 |
| HsRTL8b- | 859bp 3′UTR | terminal) | | | |
| 3′UTR | | | | | |
| FLAG- | RTL8c (AA1-113), with | 3x FLAG (N- | Homo sapiens | NP_001071639.1 | pcDNA3.1 |
| HsRTL8c- | 821bp 3′UTR | terminal) | | | |
| 3′UTR | | | | | |
| HA-HsRTL8c- | RTL8c (AA1-113), with | 2x HA (N-terminal) | Homo sapiens | NP_001071639.1 | pcDNA3.1 |
| 3′UTR | 821bp 3′UTR | | | | |
| MmRTL8b | RTL8b (AA1-113) | 3x FLAG (N- | Mus musculus | NP_001018073.1 | pcDNA3.1 |
| | | terminal) | | | |
| gag-pol flow | PEG10 (AA1-708) | Dendra2 (C- | Homo sapiens | NP_055883.2 | pDendra2 |
| reporter (Human) | | terminal), IRES-CFP | | | |
| gag-pol^{FFS} flow | PEG10 (AA1-708) | Dendra2 (C- | Homo sapiens | NP_055883.2 | pDendra2 |
| reporter (Human) | | terminal), IRES-CFP | | | |
| gag-pol flow | PEG10 (AA1-1005) | Dendra2 (C- | Mus musculus | NP_570947.2 | pDendra2 |
| reporter (Mouse) | | terminal), IRES-CFP | | | |

### REFERENCES

1. Volff, J.-N. (2006). Turning junk into gold: domestication of transposable elements and the creation of new genes in eukaryotes. BioEssays 28, 913-922. 10.1002/bies.20452.
2. Feschotte, C., and Pritham, E. J. (2007). DNA transposons and the evolution of eukaryotic genomes. Annu. Rev. Genet. 41, 331-368. 10.1146/annurev.genet.40.110405.090448.
3. Etchegaray, E., Naville, M., Volff, J.-N., and Haftek-Terreau, Z. (2021).

Transposable element-derived sequences in vertebrate development. Mob. DNA 12, 1. 10.1186/s13100-020-00229-5.
4. Ono, R., Nakamura, K., Inoue, K., Naruse, M., Usami, T., Wakisaka-Saito, N., Hino, T., Suzuki-Migishima, R., Ogonuki, N., Miki, H., et al. (2006). Deletion of Peg10, an imprinted gene acquired from a retrotransposon, causes early embryonic lethality. Nat. Genet. 38, 101-106. 10.1038/ng1699.
5. Abed, M., Verschueren, E., Budayeva, H., Liu, P., Kirkpatrick, D.S., Reja, R., Kummerfeld, S. K., Webster, J. D., Gierke, S., Reichelt, M., et al. (2019). The Gag protein PEG10 binds to RNA and regulates trophoblast stem cell lineage specification. PLOS ONE 14, e0214110. 10.1371/journal.pone.0214110.
6. Rial Verde, E. M., Lee-Osbourne, J., Worley, P. F., Malinow, R., and Cline, H.T. (2006). Increased Expression of the Immediate-Early Gene Arc/Arg3.1 Reduces AMPA Receptor-Mediated Synaptic Transmission. Neuron 52, 461-474. 10.1016/j.neuron.2006.09.031.
7. Volff, J.-N. (2009). Cellular Genes Derived from Gypsy/Ty3 Retrotransposons in Mammalian Genomes. Ann. N. Y. Acad. Sci. 1178, 233-243. 10.1111/j.1749-6632.2009.05005.x.
8. Wang, J., and Han, G.-Z. (2021). Unearthing LTR Retrotransposon gag Genes Co-opted in the Deep Evolution of Eukaryotes. Mol. Biol. Evol. 38, 3267-3278. 10.1093/molbev/msab101.
9. Wang, J., and Han, G.-Z. (2020). Frequent Retroviral Gene Co-option during the Evolution of Vertebrates. Mol. Biol. Evol. 37, 3232-3242. 10.1093/molbev/msaa180.
10. Frank, J. A., and Feschotte, C. (2017). Co-option of endogenous viral sequences for host cell function. Curr. Opin. Virol. 25, 81-89. 10.1016/j.coviro.2017.07.021.
11. Frank, J. A., Singh, M., Cullen, H. B., Kirou, R. A., Benkaddour-Boumzaouad, M., Cortes, J. L., Pérez, J. G., Coyne, C. B., and Feschotte, C. (2022). Evolution and antiviral activity of a human protein of retroviral origin. Science 378, 422-428.
12. Campillos, M., Doerks, T., Shah, P., and Bork, P. (2006). Computational characterization of multiple Gag-like human proteins. Trends Genet. 22, 585-589. 10.1016/j.tig.2006.09.006.
13. Pastuzyn, E. D., Day, C. E., Kearns, R. B., Kyrke-Smith, M., Taibi, A. V., McCormick, J., Yoder, N., Belnap, D. M., Erlendsson, S., Morado, D. R., et al. (2018). The Neuronal Gene Arc Encodes a Repurposed Retrotransposon Gag Protein that Mediates Intercellular RNA Transfer. Cell 172, 275-288.e18. 10.1016/j.cell.2017.12.024.
14. Ashley, J., Cordy, B., Lucia, D., Fradkin, L.G., Budnik, V., and Thomson, T. (2018). Retrovirus-like Gag Protein Arc1 Binds RNA and Traffics across Synaptic Boutons. Cell 172, 262-274.e11. 10.1016/j.cell.2017.12.022.
15. Segel, M., Lash, B., Song, J., Ladha, A., Liu, C. C., Jin, X., Mekhedov, S. L., Macrae, R. K., Koonin, E. V., and Zhang, F. (2021). Mammalian retrovirus-like protein PEG10 packages its own mRNA and can be pseudotyped for mRNA delivery. Science 373, 882-889. 10.1126/science.abg6155.
16. Black, H. H., Hanson, J. L., Roberts, J. E., Leslie, S. N., Campodonico, W., Ebmeier, C. C., Holling, G. A., Tay, J. W., Matthews, A. M., Ung, E., et al. (2023). UBQLN2 restrains the domesticated retrotransposon PEG10 to maintain neuronal health in ALS. eLife 12, e79452. 10.7554/eLife.79452.

17 Armezzani, A., Varela, M., Spencer, T., Palmarini, M., and Arnaud, F. (2014). “Ménage à Trois”: The Evolutionary Interplay between JSRV, enJSRVs and Domestic Sheep. Viruses 6, 4926-4945. 10.3390/v6124926.
18. Murcia, P. R., Arnaud, F., and Palmarini, M. (2007). The Transdominant Endogenous Retrovirus enJS56A1 Associates with and Blocks Intracellular Trafficking of Jaagsiekte Sheep Retrovirus Gag. J. Virol. 81, 1762-1772. 10.1128/JVI.01859-06.
19. Cottee, M. A., Beckwith, S. L., Letham, S. C., Kim, S. J., Young, G. R., Stoye, J.P., Garfinkel, D. J., and Taylor, I. A. (2021). Structure of a Tyl restriction factor reveals the molecular basis of transposition copy number control. Nat. Commun. 12, 5590. 10.1038/s41467-021-25849-0.
20. Tucker, J. M., Larango, M. E., Wachsmuth, L. P., Kannan, N., and Garfinkel, D. J. (2015). The Tyl Retrotransposon Restriction Factor p22 Targets Gag. PLOS Genet. 11, e1005571. 10.1371/journal.pgen.1005571.
21. Goff, S. P. (1996). Operating under a Gag Order: a Block against Incoming Virus by the Fvl Gene. Cell 86, 691-693. 10.1016/S0092-8674 (00) 80141-5.
22. Emerson, R. O., and Thomas, J. H. (2011). Gypsy and the Birth of the SCAN Domain. J. Virol. 85, 12043-12052. 10.1128/JVI.00867-11.
23. Chowdhury, S., Shepherd, J. D., Okuno, H., Lyford, G., Petralia, R. S., Plath, N., Kuhl, D., Huganir, R. L., and Worley, P. F. (2006). Arc/Arg3.1 Interacts with the Endocytic Machinery to Regulate AMPA Receptor Trafficking. Neuron 52, 445-459. 10.1016/j.neuron.2006.08.033.
24. Messaoudi, E., Kanhema, T., Soule, J., Tiron, A., Dagyte, G., da Silva, B., and Bramham, C.R. (2007). Sustained Arc/Arg3.1 Synthesis Controls Long-Term Potentiation Consolidation through Regulation of Local Actin Polymerization in the Dentate Gyrus In Vivo. J. Neurosci. 27, 10445-10455. 10.1523/JNEUROSCI.2883-07.2007.
25. Shiura, H., Ono, R., Tachibana, S., Kohda, T., Kaneko-Ishino, T., and Ishino, F. (2021). PEG10 viral aspartic protease domain is essential for the maintenance of fetal capillary structure in the mouse placenta. Development 148, dev199564. https://doi.org/10.1242/dev.199564.
26. Akamatsu, S., Wyatt, A. W., Lin, D., Lysakowski, S., Zhang, F., Kim, S., Tse, C., Wang, K., Mo, F., Haegert, A., et al. (2015). The Placental Gene PEG10 Promotes Progression of Neuroendocrine Prostate Cancer. Cell Rep. 12, 922-936. 10.1016/j.celrep.2015.07.012.
27. Pandya, N. J., Wang, C., Costa, V., Lopatta, P., Meier, S., Zampeta, F. I., Punt, A.M., Mientjes, E., Grossen, P., Distler, T., et al. (2021). Secreted retrovirus-like GAG-domain-containing protein PEG10 is regulated by UBE3A and is involved in Angelman syndrome pathophysiology. Cell Rep. Med. 2, 100360. 10.1016/j.xcrm.2021.100360.
28. Whiteley, A. M., Prado, M. A., de Poot, S. A. H., Paulo, J.A., Ashton, M., Dominguez, S., Weber, M., Ngu, H., Szpyt, J., Jedrychowski, M. P., et al. (2021). Global proteomics of Ubqln2-based murine models of ALS. J. Biol. Chem. 296, 100153. 10.1074/jbc.RA120.015960.
29. Baietti, M. F., Zhang, Z., Mortier, E., Melchior, A., Degeest, G., Geeraerts, A., Ivarsson, Y., Depoortere, F., Coomans, C., Vermeiren, E., et al. (2012). Syndecan-syntenin-ALIX regulates the biogenesis of exosomes. Nat. Cell Biol. 14, 677-685. 10.1038/ncb2502.
30. Brandt, J., Veith, A. M., and Volff, J.-N. (2005). A family of neofunctionalized Ty3/gypsy retrotransposon genes in mammalian genomes. Cytogenet. Genome Res. 110, 307-317. 10.1159/000084963.
31. Brandt, J., Schrauth, S., Veith, A.-M., Froschauer, A., Haneke, T., Schultheis, C., Gessler, M., Leimeister, C., and Volff, J.-N. (2005). Transposable elements as a source of genetic innovation: expression and evolution of a family of retrotransposon-derived neogenes in mammals. Gene 345, 101-111. 10.1016/j.gene.2004.11.022.
32. Mohan, H. M., Trzeciakiewicz, H., Pithadia, A., Crowley, E. V., Pacitto, R., Safren, N., Trotter, B., Zhang, C., Zhou, X., Zhang, Y., et al. (2022). RTL8 promotes nuclear localization of UBQLN2 to subnuclear compartments associated with protein quality control. Cell. Mol. Life Sci. 79, 176. 10.1007/s00018-022-04170-z.
33. Huttlin, E. L., Ting, L., Bruckner, R. J., Gebreab, F., Gygi, M. P., Szpyt, J., Tam, S., Zarraga, G., Colby, G., Baltier, K., et al. (2015). The BioPlex Network: A Systematic Exploration of the Human Interactome. Cell 162, 425-440. 10.1016/j.cell.2015.06.043.
34. Huttlin, E. L., Bruckner, R. J., Paulo, J. A., Cannon, J. R., Ting, L., Baltier, K., Colby, G., Gebreab, F., Gygi, M.P., Parzen, H., et al. (2017). Architecture of the human interactome defines protein communities and disease networks. Nature 545, 505-509. 10.1038/nature22366.
35. Klementieva, N. V., Lukyanov, K. A., Markina, N. M., Lukyanov, S. A., Zagaynova, E. V., and Mishin, A.S. (2016). Green-to-red primed conversion of Dendra2 using blue and red lasers. Chem. Commun. 52, 13144-13146. 10.1039/C6CC05599K.
36. Golda, M., Motyan, J. A., Mahdi, M., and Tozser, J. (2020). Functional Study of the Retrotransposon-Derived Human PEG10 Protease. Int. J. Mol. Sci. 21, 2424. 10.3390/ijms21072424.
37. Clark, M. B., Jänicke, M., Gottesbühren, U., Kleffmann, T., Legge, M., Poole, E. S., and Tate, W.P. (2007). Mammalian Gene PEG10 Expresses Two Reading Frames by High Efficiency-1 Frameshifting in Embryonic-associated Tissues. J. Biol. Chem. 282, 37359-37369. 10.1074/jbc.M705676200.
38. Li, S., Hill, C. P., Sundquist, W. I., and Finch, J. T. (2000). Image reconstructions of helical assemblies of the HIV-1 CA protein. Nature 407, 409-413. 10.1038/35030177.
39. Ganser-Pornillos, B. K., Yeager, M., and Sundquist, W. I. (2008). The structural biology of HIV assembly. Curr. Opin. Struct. Biol. 18, 203-217. 10.1016/j.sbi.2008.02.001.
40. Dodonova, S. O., Prinz, S., Bilanchone, V., Sandmeyer, S., and Briggs, J. A. G. (2019). Structure of the Ty3/Gypsy retrotransposon capsid and the evolution of retroviruses. Proc. Natl. Acad. Sci. 116, 10048-10057. 10.1073/pnas.1900931116.
41. Zhao, G., Perilla, J. R., Yufenyuy, E. L., Meng, X., Chen, B., Ning, J., Ahn, J., Gronenborn, A.M., Schulten, K., Aiken, C., et al. (2013). Mature HIV-1 capsid structure by cryo-electron microscopy and all-atom molecular dynamics. Nature 497, 643-646. 10.1038/nature12162.
42. Briggs, J. A. G., Riches, J. D., Glass, B., Bartonova, V., Zanetti, G., and Kräusslich, H.-G. (2009). Structure and assembly of immature HIV. Proc. Natl. Acad. Sci. 106, 11090-11095. 10.1073/pnas.0903535106.
43. Zurowska, K., Alam, A., Ganser-Pornillos, B. K., and Pornillos, O. (2022). Structural evidence that MOAP1 and PEG10 are derived from retrovirus/retrotransposon Gag proteins. Proteins Struct. Funct. Bioinforma. 90, 309-313. 10.1002/prot.26204.
44. Kozak, C. A., and Chakraborti, A. (1996). Single Amino Acid Changes in the Murine Leukemia Virus Capsid Protein Gene Define the Target ofFv1Resistance. Virology 225, 300-305. 10.1006/viro.1996.0604.
45. Stevens, A., Bock, M., Ellis, S., LeTissier, P., Bishop, K. N., Yap, M. W., Taylor, W., and Stoye, J.P. (2004). Retroviral Capsid Determinants of Fvl NB and NR Tropism. J. Virol. 78, 9592-9598. 10.1128/JVI.78.18.9592-9598.2004.
46. Uhlen, M., Fagerberg, L., Hallström, B.M., Lindskog, C., Oksvold, P., Mardinoglu, A., Sivertsson, A., Kampf, C., Sjöstedt, E., Asplund, A., et al. (2015). Tissue-based map of the human proteome. Science 347, 1260419. 10.1126/science.1260419.
47. Yufenyuy, E. L., and Aiken, C. (2013). The NTD-CTD intersubunit interface plays a critical role in assembly and stabilization of the HIV-1 capsid. Retrovirology 10, 29. 10.1186/1742-4690-10-29.
48. Passerini, L. D., Keckesova, Z., and Towers, G.J. (2006). Retroviral Restriction Factors Fv1 and TRIM5a Act Independently and Can Compete for Incoming Virus before Reverse Transcription. J VIROL 80, 2100-2105.
49. Fernandopulle, M. S., Prestil, R., Grunseich, C., Wang, C., Gan, L., and Ward, M. E. (2018). Transcription Factor-Mediated Differentiation of Human iPSCs into Neurons: Rapid differentiation of iPSCs into neurons. Curr. Protoc. Cell Biol. 79, e51. 10.1002/cpcb.51.
50. Madeira, F., Pearce, M., Tivey, A. R. N., Basutkar, P., Lee, J., Edbali, O., Madhusoodanan, N., Kolesnikov, A., and Lopez, R. (2022). Search and sequence analysis tools services from EMBL-EBI in 2022. Nucleic Acids Res. 50, W276-W279. 10.1093/nar/gkac240.
51. Sievers, F., Wilm, A., Dineen, D., Gibson, T. J., Karplus, K., Li, W., Lopez, R., Mc William, H., Remmert, M., Söding, J., et al. (2011). Fast, scalable generation of high-quality protein multiple sequence alignments using Clustal Omega. Mol. Syst. Biol. 7, 539. 10.1038/msb.2011.75.
52. Kelley, L. A., Mezulis, S., Yates, C. M., Wass, M. N., and Sternberg, M. J. E. (2015). The Phyre2 web portal for protein modeling, prediction and analysis. Nat. Protoc. 10, 845-858. 10.1038/nprot.2015.053.
53. Pettersen, E. F., Goddard, T. D., Huang, C. C., Couch, G. S., Greenblatt, D. M., Meng, E. C., and Ferrin, T. E. (2004). UCSF Chimera--A visualization system for exploratory research and analysis. J. Comput. Chem. 25, 1605-1612. 10.1002/jcc.20084.
54. Jumper, J., Evans, R., Pritzel, A., Green, T., Figurnov, M., Ronneberger, O., Tunyasuvunakool, K., Bates, R., Žídek, A., Potapenko, A., et al. (2021). Highly accurate protein structure prediction with AlphaFold. Nature 596, 583-589. 10.1038/s41586-021-03819-2.

1. A method of increasing production of virus-like particles (VLPs) in a cell, comprising the step of disrupting the activity or expression of RTL8, UBQLN2, or RTL8 and UBOLN2 in the cell, wherein said disruption causes an increase in PEG10 VLP formation.

2. The method of claim 1, wherein said cell comprises a human cell.

2. The method of claim 1, wherein said RTL8 comprises a nucleotide sequence selected from SEQ ID NO.'s 1-3, or a fragment or variant thereof, and wherein said UBQLN2 comprises a nucleotide sequence according to SEQ ID NO. 9, or a fragment or variant thereof.

3. The method of claim 1, wherein said RTL8 comprises an amino acid sequence selected from SEQ ID NO.'s 4-7, or a fragment or variant thereof, and wherein said UBQLN2 comprises an amino acid sequence according to SEQ ID NO. 10, or a fragment or variant thereof.

4. The method of claim 1, further comprising the step of expressing a heterologous nucleotide sequence, operably linked to a promoter, encoding a target mRNA, or gene editing RNA or peptide.

5. The method of claim 4, wherein said mRNA or gene editing RNA or peptide comprises a therapeutic mRNA that is configured to be coupled with said PEG10 VLP.

6. The method of claim 1, wherein said step of disrupting the expression of and/or activity of RTL8 and/or UBQLN2 comprises the step of contacting the cell with a therapeutically effective amount of an agent that inhibits the activity or expression of RTL8, and/or UBQLN2, or homologs of RTL8, and UBQLN2.

7. The method of claim 6, wherein said agent is selected from the group consisting of: a nucleic acid molecule, an antibody or a biologically active fragment thereof, and an aptamer.

8. The method of claim 1, wherein said nucleic acid molecule is selected from the group consisting of: an anti-sense oligonucleotide, an inhibitory RNA molecule, a DNA enzyme, and a ribozyme that specifically inhibits the expression or activity of RTL8, UBQLN2, or homologs of RTL8, and UBQLN2.

9. The method of claim 1, wherein said step of disrupting the expression of and/or activity of RTL8 comprises the step of genetically modifying the cell to knock-out of knock-down RTL8, or UBQLN2 or homologs of RTL8, and UBOLN2.

10. The method of claim 1, wherein said step of disrupting the expression of and/or activity of RTL8 comprises the step of genetically modifying the cell to generate one or mutations in RTL8, or UBQLN2 that decrease or inhibit its expression or activity.

11. A PEG10 VLP produced by the method of claim 1.

12. The PEG10 VLP of claim 11, wherein said PEG10 VLP is isolated.

13-56. (canceled)


SEQUENCE LISTING (10 sequences)

SEQ ID NO: 1 - DNA, 1204 bp, Homo sapiens
  source 1..1204: mol_type = genomic DNA
gaaactccgcggagcgcgcgcggcacgatggacggtcgggtgcagctgatgaaggccctcctggccgggcccctccggcccgcggcgcgtcgctggaggaacccgattccctttcccgagacgtttgacggagataccgaccgactcccggagttcatcgtgcagacgagctcctacatgttcgtggacgagaacacgttctccaacgacgccctgaaggtgacgttcctcatcacccgcctcacggggccagccctgcagtgggtgatcccctacatcaggaaggagagccccctgctcaatgattaccggggcttcctggccgagatgaagcgggtctttggatgggaggaggacgaggacttctaggccgggagacccttgggcctgggggcgggtgctctgggaagagttcgctgtgccagtggccaccgctagggtctccacaggcgccctccctccgcgcctccctccccctcgagccgccgcgatgtcccctgcgctcctgtcccctcccgcgtagtgcttgcctttgttccaggaatagcgctccaggctcctgctgcagctcctgggccgcactctagagcgcgctgcctccgccctcttttgccagcccagcccctccatgcacatttggacgctgtcctgcacttcaggtgcaagctgggctcctgttacatactggacagaccacccacagccgctgctgccaacccacctccactcctccagactgccagccaactacatcattctgcccacagacctacgctaccactgccatcgccatccaccgcatctcaccaacagactgtggctcctagtgatgtggactcacctcggaggtatccgagctggacacagcccctggacagtgatccagacagctggccgtcccccaaggagcctgtcaccttcagcgagacccatttcctccccatccccagagacctcttgtgttcctgccacatagctgccagggcttaagtgtgcctggcaaccaaatcgaatctctcattttctcctgtggaccagttagttttgcctagaatcctgttttcttctgaatttgcagctgtctctctgatgggtgccttttgttcaacacagtaagccctgctcccttccctgctctaatacactacctgtacaaaggttttttccttatttttaataaatgtcagacactattaaatagaaa

SEQ ID NO: 2 - DNA, 2030 bp, Homo sapiens
  source 1..2030: mol_type = genomic DNA
atgcacttcccgctacgcctcggtgcagcccccggcgaagcccagcgcagctcagctaaactcagcggagccagcgcggcgcgatggaaggtcgagtgcagctgatgaaggccctcctggctcggcccctccggcccgcggcgcgtcgctggaggaacccgattccctttcccgagacgtttgatggcgataccgaccggctcccggagttcatcgtgcagacgagctcctacatgttcgtggacgagaacacgttctccaacgacgccctgaaggtgacgttcctcatcacccgcctcacggggcccgccctgcagtgggtgatcccctacatcaagaaggagagccccctcctcagtgattaccggggcttcctggctgagatgaagcgggtctttggatgggaggaggacgaggacttctaggccgggagaccctcgggcctggggaccggtgctctgggaagggtccgctgtgttactggccgccgccagggtcgccaccggcgccctccctccgcgcctccctccccctcgagccgccacgatgtcccctgcgctcctgtcccctcccgcgtagtgcttgcctttgttccaggaatagcgctccaggctcctgctgccgcccctgggcctcactctggagcgggctgcctccgccctctcgtgccagctcagcccctcccatgcacatttggacgctgtcctgcgctccagttgcacaaagctgagctcctgttacacactgacagaccactgacagccgccgctgccaacccccctcctcctcaccagacttccagacgactacatcatgctgcccacagacctgcgctgccactgccatcgccatccatcgcattccaccgacagactgctgcttctagtgatctgtactcacctcggaggtatctgggctggacacagcccctggacaatgatccagacagctggctgcccctcaagggacctgttaccttcagcgagacccatttcctccccaatcccagagactttttgtgctcctgccacatggctgccagggctcagttgtgcctggcagccaaatcgagtctctcattttctcttgtggaccagttagttttgcccagaatccatttctcttctgaatttgcagcattttctcaggtgctttttgatcaacaaaataacttgtatcttctgcttaactcctttatactacctggagaaaagtattttccatattttcaataaatgtcagacactattgagaaagaaatagagtctgaatgaatatggcacctttccctccctggtcctgtaaatgtggtccatatcttgttcagaagcgatatcccctgccattcagtgactgatgttgactcctgcattataaaacatctaagatagagtcctgtggtaacctgggggatagtgtccccactggtgatccttctgtcccagatgttgaatcctctaaaccagttaggattatcccaagcctcttagtaaatgaatcgttacctcattttggtcaacactatgtgagaagtcagccagcaggctcttggaaaatgtatcctcatttccagaaagtggtttgaaaacacaattcccaaattgttacaaaccataaatcccacacagattattggtgaacagctgtaaaattttactcaacccatgattcagggaaccaacagaattatataggtgcttttcaaaagcatttataaattcttgattatgtaggtagtggggggaaatgcagaaatgagatcattaatttagatacaaacctggttgatgaccagggggttttattttgcagaagaattgggtatttggtatcattttattcaattacatgtgatcctggtacatttcataagcttgggtaaaaaaattttttgttattttccatgcacaataagggaaatgttttaattggctttgtgcctcctgctttacaacagttctggcctcaagttaccaggtagcctgaactttgattttattttaccattaaattgctttaataccattgta

SEQ ID NO: 3 - DNA, 1192 bp, Homo sapiens
  source 1..1192: mol_type = genomic DNA
attgacgtccagcgaagcgaggagcagcgatggacggtcgggtgcagctgataaaggccctcctggccttgccgatccggcctgcgacgcgtcgctggaggaacccgattccctttcccgagacgtttgacggcgataccgaccgactcccggagttcatcgtgcagacgggctcctacatgttcgtggacgagaacacgttctccagcgacgccctgaaggtgacgttcctcatcacccgcctcacagggcccgccctgcagtgggtgatcccctacatcaagaaggagagccccctcctcaatgattaccggggctttctggccgagatgaagcgagtctttggatgggaggaggacgaggacttctaggccgggagaccctcgggcctgggggcgggtgctctggggagggtccgctgtgttactggccgccgccagggtcgccaccggcgccctccctccgcgcctccctccccctcgagccgccgcgatgtcccctgcgctcctgttccctcccgcgtagtgcttgcctttgttccaggaatagcgctccaggctcctgctgccgcccctgggcctcactctggagcgagccgccgccctctccttccagccagccagcccctcccatgtacatttggacgctgtcctgcgctccagctgcaagctgggctcctgttacacactggacagaccacccactgccgccgctgccaagccctctcctccccaccagactgccagacgactacatcattctgcccacagacctgcgctgccacagccatcgccatccatcgcatcccaccgacagactgctgctcctagtgatctggactcacctcggaggtatctgggctggccacagtccctggacagtgatccagacagctggccgccccccaagggatctgtcaccttcagcgagacctatttcctccccacccccagaaacctcttgtgttcttgcctaggcccaggtgttcctggcagccaaatcgagtctctcattttctcttgtggaccagttagttttgcccataacgcagtattctgagtttgcaactgtctctctgatgtgtgccttttgttcaacacagtaacccctgcattctgctctgctctaatacactacctggagaaagtcttttccttattttcaataaatgtcagacattattgaaaagaaa

SEQ ID NO: 4 - protein, 113 aa, Homo sapiens
MDGRVQLMKALLAGPLRPAARRWRNPIPFPETFDGDTDRLPEFIVQTSSYMFVDENTFSNDALKVTFLITRLTGPALQWVIPYIRKESPLLNDYRGFLAEMKRVFGWEEDEDF

SEQ ID NO: 5 - protein, 83 aa, Homo sapiens
MDGRVQLMKALLAGPLRPAARRWRNPIPFPETFDGDTDRLPEFIVQTSSYMFVDENTFSNDALKTYATTAIAIHRISPTDCGS

SEQ ID NO: 6 - protein, 113 aa, Homo sapiens
MEGRVQLMKALLARPLRPAARRWRNPIPFPETFDGDTDRLPEFIVQTSSYMFVDENTFSNDALKVTFLITRLTGPALQWVIPYIKKESPLLSDYRGFLAEMKRVFGWEEDEDF

SEQ ID NO: 7 - protein, 113 aa, Homo sapiens
MDGRVQLIKALLALPIRPATRRWRNPIPFPETFDGDTDRLPEFIVQTGSYMFVDENTFSSDALKVTFLITRLTGPALQWVIPYIKKESPLLNDYRGFLAEMKRVFGWEEDEDF

SEQ ID NO: 8 - RNA, 19 nt, synthetic construct
  source 1..19: mol_type = other RNA
gctcctacatgttcgtgga

SEQ ID NO: 9 - DNA, 1875 bp, Homo sapiens
  source 1..1875: mol_type = genomic DNA
atggctgagaatggcgagagcagcggccccccgcgcccctcccgcggccctgctgcggcccaaggctcggctgctgccccggctgagcctaaaatcatcaaagtcacggtgaagactcccaaagagaaagaggagttcgcggtgcccgagaacagctcggttcagcagtttaaggaagcgatttcgaaacgcttcaaatcccaaaccgatcagctagtgctgatttttgccggaaaaatcttaaaagatcaagataccttgatccagcatggcatccatgatgggctgactgttcaccttgtcatcaaaagccagaaccgacctcagggccagtccacgcagcctagcaatgccgcgggaactaacactacctcggcgtcgactcccaggagtaactccacacctatttccacaaatagcaacccgtttgggttggggagcctgggaggacttgcaggccttagcagcctgggcttgagctcgaccaacttctctgagctccagagccagatgcagcagcagcttatggccagccctgagatgatgatccaaataatggaaaatccctttgttcagagcatgctttcgaatcccgatctgatgaggcagctcattatggctaatccacagatgcagcaattgattcagagaaacccagaaatcagtcacctgctcaacaacccagacataatgaggcagacactcgaaattgccaggaatccagccatgatgcaagagatgatgagaaatcaagacctggctcttagcaatctagaaagcatcccaggtggctataatgctttacggcgcatgtacactgacattcaagagccgatgctgaatgccgcacaagagcagtttgggggtaatccatttgcctccgtggggagtagttcctcctctggggaaggtacgcagccttcccgcacagaaaatcgcgatccactacccaatccatgggcaccaccgccagctacccagagttctgcaactaccagcacgaccacaagcactggtagtgggtctggcaatagttccagcaatgctactgggaacaccgttgctgccgctaattatgtcgccagcatctttagtaccccaggcatgcagagcctgctgcaacagataactgaaaacccccagctgattcagaatatgctgtcggcgccctacatgagaagcatgatgcagtcgctgagccagaatccagatttggctgcacagatgatgctgaatagcccgctgtttactgcaaatcctcagctgcaggagcagatgcggccacagctcccagccttcctgcagcagatgcagaatccagacacactatcagccatgtcaaacccaagagcaatgcaggctttaatgcagatccagcaggggctacagacattagccactgaagcacctggcctgattccgagcttcactccaggtgtgggggtgggggtgctgggaaccgctataggccctgtaggcccagtcacccccataggccccataggccctatagtcccttttacccccataggccccattgggcccataggacccactggccctgcagccccccctggctccaccggctctggtggccccacggggcctactgtgtccagcgctgcacctagtgaaaccacgagtcctacatcagaatctggacccaaccagcagttcattcagcaaatggtgcaggccctggctggagcaaatgctccacagctgccgaatccagaagtcagatttcagcaacaactggaacagctcaacgcaatggggttcttaaaccgtgaagcaaacttgcaggccctaatagcaacaggaggcgacatcaatgcagccattgaaaggctgctgggctcccagccatcgtaa

SEQ ID NO: 10 - protein, 624 aa, Homo sapiens
MAENGESSGPPRPSRGPAAAQGSAAAPAEPKIIKVTVKTPKEKEEFAVPENSSVQQFKEAISKRFKSQTDQLVLIFAGKILKDQDTLIQHGIHDGLTVHLVIKSQNRPQGQSTQPSNAAGTNTTSASTPRSNSTPISTNSNPFGLGSLGGLAGLSSLGLSSTNFSELQSQMQQQLMASPEMMIQIMENPFVQSMLSNPDLMRQLIMANPQMQQLIQRNPEISHLLNNPDIMRQTLEIARNPAMMQEMMRNQDLALSNLESIPGGYNALRRMYTDIQEPMLNAAQEQFGGNPFASVGSSSSSGEGTQPSRTENRDPLPNPWAPPPATQSSATTSTTTSTGSGSGNSSSNATGNTVAAANYVASIFSTPGMQSLLQQITENPQLIQNMLSAPYMRSMMQSLSQNPDLAAQMMLNSPLFTANPQLQEQMRPQLPAFLQQMQNPDTLSAMSNPRAMQALMQIQQGLQTLATEAPGLIPSFTPGVGVGVLGTAIGPVGPVTPIGPIGPIVPFTPIGPIGPIGPTGPAAPPGSTGSGGPTGPTVSSAAPSETTSPTSESGPNQQFIQQMVQALAGANAPQLPNPEVRFQQQLEQLNAMGFLNREANLQALIATGGDINAAIERLLGSQPS